(19) United States
(12) Patent Application Publication
(10) Pub. No.: US 2011/0251175 A1
(43) Pub. Date: Oct. 13, 2011

(54) Stigmine Conjugates for Substance Use Disorders

(76) Inventors: Nadia Rupniak, Cary, NC (US); James F. White, Carlisle, MA (US)

(21) Appl. No.: 13/003,721

(22) PCT Filed: Jul. 16, 2009
(86) PCT No.: PCT/US09/50851

§ 371 Date: Jun. 28, 2011

Related U.S. Application Data
(60) Provisional application No. 61/135,139, filed on Jul. 16, 2008.

(51) Int. Cl. A61K 31/27; A61K 31/4525; A61K 31/445; A61P 9/00; A61K 31/553; A61P 25/30; A61P 25/36; A61P 25/00; A61K 31/55; A61K 31/381

(52) U.S. Cl. 514/211.13; 514/486; 514/487; 514/217; 514/488; 514/321; 514/330; 514/438

(57) ABSTRACT

The invention relates to methods for the treatment or prevention of substance use disorders.

### BACKGROUND OF THE INVENTION

Substance use disorders such as drug addiction are characterized by compulsive, at times uncontrollable, drug craving, seeking, and use that persists even in the face of life-threatening consequences. Addicts experience unpleasant physical and psychological symptoms if they discontinue the drug, which makes abstinence difficult. Drug addiction is a chronic illness, with relapses possible even after long periods of abstinence. Addiction may expose people to increased risk for other illnesses brought on by poor health habits (e.g. AIDS), or because of toxic effects of the drugs themselves (e.g. lung cancer, cirrhosis) (Principles of Drug Addiction Treatment: A Research Based Guide, National Institute on Drug Abuse). Brain imaging studies reveal that addiction involves alterations in cognitive function, notably impairment of executive functions of the frontal cortex, that may maintain drug use despite punishment for such behavior (Carpenter S. (2001) Monitor on Psychology, Vol. 32, No. 5. Cognition is central to drug addiction; Giancola P R, Moss H B (1998). Executive cognitive functioning in alcohol use disorders. In: Galanter, M., ed. Recent Developments in Alcoholism: Volume 14. The Consequences of Alcoholism. New York: Plenum Press, pp. 227-251).

A large number of legal and illicit drugs and prescription medicines have abuse liability. These include stimulants (e.g., amphetamine, methamphetamine, cocaine), benzodizepines (e.g. diazepam, temazepam), opioids (e.g., morphine, fentanyl, heroin), nicotine (cigarettes) and alcohol (e.g., beer, wine, spirits).

A number of effective pharmacotherapies for addiction have been introduced and illustrate the postential usefulness of medications for drug abuse treatment. One approach is to administer a long-acting substitute drug at a sufficient dose to prevent withdrawal, block the reinforcing effects of the abused drug, and decrease craving (e.g., methadone for opiate addicts, transdermal nicotine patches for smokers). An alternative approach has been identified as a result of treating other co-morbid symptoms, most notably depression, frequently seen in addicts. Various antidepressant drugs have been shown to be effective as pharmacotherapy to support smoking cessation and alcohol abstinence (Hurt R, Sachs D, Glover, E. Offord K, Johnston J, Dale L, Sullivan P (1997). A comparison of sustained-release bupropion and placebo for smoking cessation. New England Journal of Medicine 337: 1195; Hughes J, Stead L, Lancaster T (2004). Antidepressants for smoking cessation. Cochrane Database Syst Rev. 2004: CD000031; Patten, C A (2002). Treating Alcoholic Smokers Who Have a History of Depression. Alcoholism: Clinical and Experimental Research 26: 1947-1949. Antidepressants may also be of value in treatment of other addictions. Medications such as antidepressants may be critical for treatment success when patients have co-morbid psychiatric disorders, such as depression, anxiety disorder, bipolar disorder, or psychosis (Principles of Drug Addiction Treatment: A Research Based Guide, National Institute on Drug Abuse).

A need exists to identify compounds which effectively treat or prevent substance use disorders.

### SUMMARY OF THE INVENTION

The present invention relates to the treatment or prevention of substance use disorders by the administration of selected bifunctional stigmines.

The invention relates to a method for the treatment or prevention of a substance use disorder comprising administering to an individual a compound having the formula:

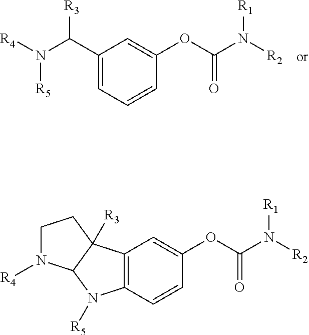

or a salt thereof. In the formula above, the variables R₁, R₂, R₃, R₄, and R₅ can be selected from the respective groups of chemical moieties later defined in the detailed description. In certain embodiments, the invention relates to the use of the compounds described herein for the manufacture of a medicament for the treatment or prevention of a substance use disorder.

In one aspect, stigmines conjugated with sympathomimetics are therapeutically useful to treat addiction by delivering sympathomimetics to the CNS that may substitute for drugs of abuse to prevent withdrawal, block the reinforcing effects of the abused drug, and/or decrease craving. The stigmines conjugated with stimulants have lower abuse potential than stimulants themselves by virtue of their accompanying acetylcholinesterase inhibition, since cholinergic side effects such as nausea would be dose-limiting. Moreover, cognitive enhancement conferred by acetylcholinestease inhibition would be beneficial to alleviate the cognitive impairment accompanying addiction. Such compounds include, but are not limited to, s-riva-1-amphetamine, s-riva-d-amphetamine, s-riva-l-methamphetamine, s-riva-d-methamphetamine, physo-d-amphetamine and s-riva-methoxyphenamine.

In another aspect, stigmines conjugated with antidepressant drugs are therapeutically useful to support abstinence from drugs of abuse by delivering to the CNS an effective antidepressant drug combined with an acetylcholinesterase inhibitor. The antidepressant efficacy is beneficial to support abstinence and treat co-morbid psychiatric symptoms, and the memory enhancement is of benefit for accompanying cognitive symptoms. Such compounds include, but are not limited to, S-riva-atomoxetine, S-riva-amoxapine, S-riva-desipramine, S-riva-nortriptyline, S-riva-protriptyline, S-riva-fluoxetine, S-riva-fluvoxamine, S-riva-paroxetine and S-riva-duloxetine.

### BRIEF DESCRIPTION OF THE DRAWINGS

FIG. 1 is two graphs which show the data from a methamphetamine drug discrimination test in rats. The rats are trained to respond on one lever when they perceive that they have been pretreated with methamphetamine, and on a second lever when they perceive that they have been pretreated with the vehicle. The data in the top figure show that as the dose of methamphetamine increases, the rats switch their responding from the vehicle-associated lever to the methamphetamine associated lever, i.e. at higher doses of methamphetamine, the rats detected that they had been injected with methamphetamine. In contrast, the data with compound 7 (S-riva-L-methamphetamine) showed that the animals never perceived an effect they associated with methamphetamine, i.e., they continued to respond primarily on the vehicle associated lever over the entire dose range. The data in the lower figure show that compound 7 was tested over a behaviorally active dose range because increasing doses caused a dose-related decrease in the rate of responding, and that higher doses could not, therefore, be tested.

FIG. 2 shows data also from a methamphetamine drug discrimination test in rats. The data in the top figure shows that a dose of 1.0 mg/kg of compound 7 shifted the methamphetamine dose-response curve to the right. These data indicate that compound 7 could block the discriminative effects of methamphetamine, and this was accomplished without behavioral disruption as shown in the lower figure. These data suggest that compound 7 may be an effective treatment for methamphetamine-like stimulant abuse.

### DETAILED DESCRIPTION OF THE INVENTION

The features and other details of the invention, either as steps of the invention or as combinations of parts of the invention, will now be more particularly described and pointed out in the claims. It will be understood that the particular embodiments of the invention are shown by way of illustration and not as limitations of the invention. The principle features of this invention can be employed in various embodiments without departing from the scope of the invention.

One aspect of the invention includes a method for the treatment or prevention of a substance use disorder comprising administering to an individual a compound having the formula:

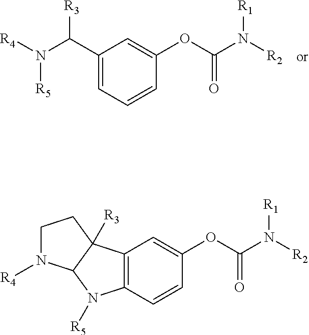

or a salt thereof, wherein R₁ is selected from the group consisting of hydrogen, unsubstituted alkyl, and substituted alkyl. R₂ is selected from the group consisting of substituted alkyl, unsubstituted aralkyl, substituted aralkyl, unsubstituted (heterocycle)alkyl, substituted (heterocycle)alkyl, unsubstituted heteroaralkyl, substituted heteroaralkyl, unsubstituted aryl, substituted aryl, unsubstituted heteroaryl, substituted heteroaryl, unsubstituted cycloalkyl, substituted cycloalkyl, unsubstituted heterocycloalkyl and substituted heterocycloalkyl; or taken together with the nitrogen atom to which they are attached, R₁ and R₂ form a 5- or 6-membered ring, further wherein the ring is substituted or unsubstituted. R₃ is selected from the group consisting of hydrogen, unsubstituted alkyl, and substituted alkyl. R₄ is selected from the group consisting of hydrogen, unsubstituted alkyl, and substituted alkyl. R₅ is selected from the group consisting of hydrogen, unsubstituted alkyl, and substituted alkyl.

In one aspect, the invention includes a method for the treatment or prevention of a substance use disorder comprising administering to an individual a compound or salt thereof having the formula

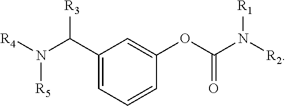

In one aspect, the invention includes a method for the treatment or prevention of a substance use disorder comprising administering to an individual a compound or salt thereof having the formula

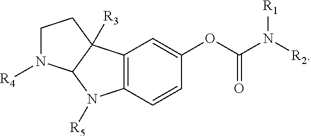

In one aspect, the invention includes a method for the treatment or prevention of a substance use disorder comprising administering to an individual a compound, wherein at least one of R₃, R₄, and R₅ is unsubstituted alkyl. In another aspect, the invention includes a method for the treatment or prevention of a substance use disorder comprising administering to an individual a compound or salt thereof, wherein at least two R₃, R₄, and R₅ are unsubstituted alkyl. In another aspect, the invention includes a method for the treatment or prevention of a substance use disorder comprising administering to an individual a compound or salt thereof, wherein R₃, R₄, and R₅ are each unsubstituted alkyl. In another aspect, the invention includes a method for the treatment or prevention of a substance use disorder comprising administering to an individual a compound or salt thereof, wherein unsubstituted alkyl is methyl.

In one aspect, the invention includes a method for the treatment or prevention of a substance use disorder comprising administering to an individual a compound or salt thereof, wherein the configuration of the stereocenter to which R₃ is attached is in the S-configuration as shown below:

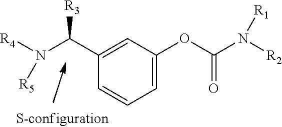

In another aspect, the invention includes a method for the treatment or prevention of a substance use disorder comprising administering to an individual a compound or salt thereof, wherein the configuration of the stereocenter to which R₃ is attached is in the R-configuration as shown below:

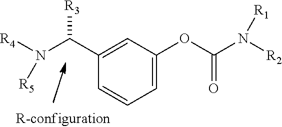

In one aspect, the invention includes a method for the treatment or prevention of a substance use disorder comprising administering to an individual a compound or salt thereof, wherein the configuration of the stereocenter to which R₃ is attached is shown below:

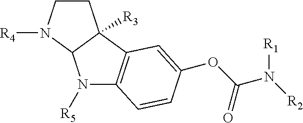

In one aspect, the invention includes a method for the treatment or prevention of a substance use disorder comprising administering to an individual a compound or salt thereof, wherein R₁ is hydrogen.

In one aspect, the invention includes a method for the treatment or prevention of a substance use disorder comprising administering to an individual a compound or salt thereof, wherein R₁ is unsubstituted alkyl. In another aspect, the invention includes a method for the treatment or prevention of a substance use disorder comprising administering to an individual a compound or salt thereof, wherein R₁ is methyl.

In one aspect, the invention includes a method for the treatment or prevention of a substance use disorder comprising administering to an individual a compound or salt thereof, wherein R₁ is substituted alkyl. In another aspect, the invention includes a method for the treatment or prevention of a substance use disorder comprising administering to an individual a compound or salt thereof, wherein R₁ is alkyl substituted with alkynyl. In another aspect, the invention includes a method for the treatment or prevention of a substance use disorder comprising administering to an individual a compound or salt thereof, where R₁ is

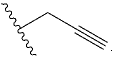

In one aspect, the invention includes a method for the treatment or prevention of a substance use disorder comprising administering to an individual a compound or salt thereof, wherein R₁ and R₂ taken together with the nitrogen atom to which they are attached form a 5- or 6-membered ring.

In one aspect, the invention includes a method for the treatment or prevention of a substance use disorder comprising administering to an individual a compound or salt thereof, wherein R₁ and R₂ taken together with the nitrogen atom to which they are attached form a 5-membered ring.

In one aspect, the invention includes a method for the treatment or prevention of a substance use disorder comprising administering to an individual a compound or salt thereof, wherein R₁ and R₂ taken together with the nitrogen atom to which they are attached form a 6-membered ring. In one aspect, the invention includes a method for the treatment or prevention of a substance use disorder comprising administering to an individual a compound or salt thereof, wherein the 6-membered ring formed by R₁ and R₂ is substituted with at least one substituent. In another aspect, the invention includes a method for the treatment or prevention of a substance use disorder comprising administering to an individual a compound or salt thereof, where the 6-membered ring formed by R₁ and R₂ is substituted with at least two substituents. In another aspect, the invention includes a method for the treatment or prevention of a substance use disorder comprising administering to an individual a compound or salt thereof, wherein the 6-membered ring formed by R₁ and R₂ is selected from the group consisting of piperidine and piperazine. In another aspect, the invention includes a method for the treatment or prevention of a substance use disorder comprising administering to an individual a compound or salt thereof, wherein the 6-membered ring formed by R₁ and R₂ is substituted at the 2-position e.g.,

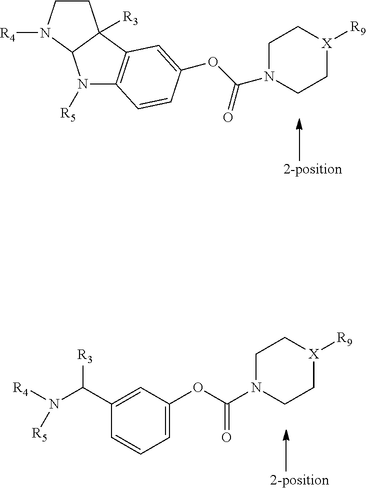

In another aspect, the invention includes a method for the treatment or prevention of a substance use disorder, comprising administering to an individual a compound or salt thereof, wherein the 6-membered ring formed by R₁ and R₂ is substituted at the 4-position e.g.,

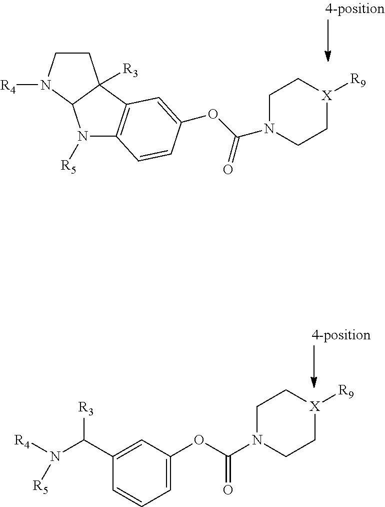

In another aspect, the invention includes a method for the treatment or prevention of a substance use disorder comprising administering to an individual a compound or salt thereof, wherein the ring formed by R₁ and R₂ is substituted with a moiety containing at least one aromatic ring. In another aspect, the invention includes a method for the treatment or prevention of a substance use disorder comprising administering to an individual a compound or salt thereof, wherein the ring formed by R₁ and R₂ is substituted with a moiety selected from

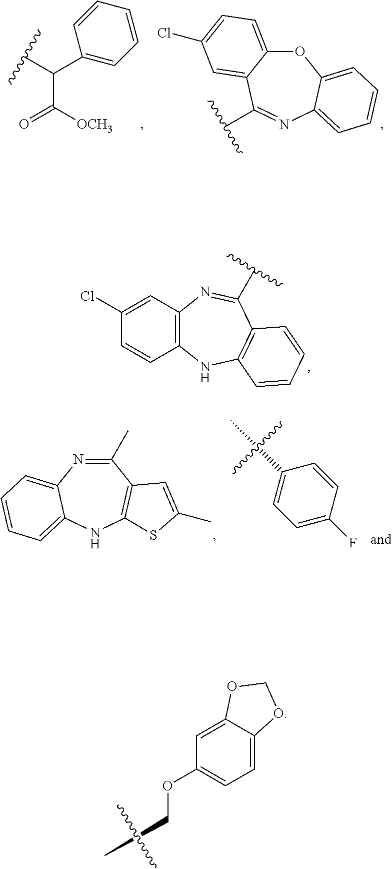

In another aspect, the invention includes a method for the treatment or prevention of a substance use disorder comprising administering to an individual a compound or salt thereof, wherein the ring formed by R₁ and R₂ is substituted with

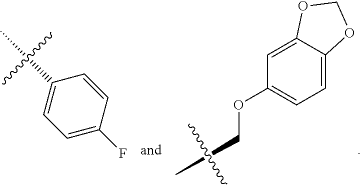

In another aspect, the invention includes a method for the treatment or prevention of a substance use disorder comprising administering to an individual a compound or salt thereof, wherein the ring formed by R₁ and R₂ is substituted with a tricyclic ring. In another aspect, the invention includes a method for the treatment or prevention of a substance use disorder comprising administering to an individual a compound or salt thereof, wherein the ring formed by R₁ and R₂ is substituted with a tricyclic ring is selected from

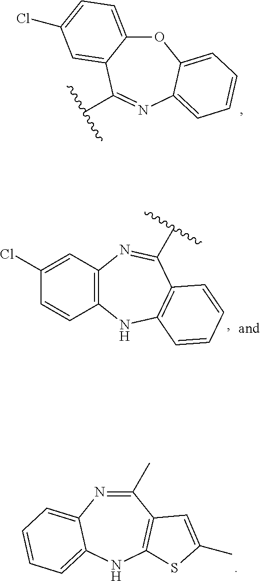

In one aspect, the invention includes a method for the treatment or prevention of a substance use disorder comprising administering to an individual a compound or salt thereof, wherein R₂ is selected from the group consisting of aralkyl, cycloalkyl, alkyl, and heteroaralkyl, further wherein R₂ is optionally substituted. In one aspect, the invention includes a method for the treatment or prevention of a substance use disorder comprising administering to an individual a compound or salt thereof, wherein the alkyl moiety of aralkyl, alkyl, and heteroaralkyl is 2 carbon atoms in length. In another aspect, the invention includes a method for the treatment or prevention of a substance use disorder comprising administering to an individual a compound or salt thereof, wherein the alkyl moiety of aralkyl, alkyl, and heteroaralkyl is 3 carbon atoms in length.

In one aspect, the invention includes a method for the treatment or prevention of a substance use disorder comprising administering to an individual a compound or salt thereof, wherein R₂ is substituted with substituted alkyl, unsubstituted alkyl, substituted cycloalkyl, unsubstituted cycloalkyl, substituted aryl, unsubstituted aryl, substituted tricyclic ring, unsubstituted tricyclic ring, substituted alkenyl-tricyclic ring, unsubstituted alkenyl-tricyclic ring, unsubstituted aryloxy, substituted aryloxy, unsubstituted oxime, and substituted oxime.

In one aspect, the invention includes a method for the treatment or prevention of a substance use disorder comprising administering to an individual a compound or salt thereof, wherein R₂ is substituted aralkyl, In one aspect, the invention includes a method for the treatment or prevention of a substance use disorder comprising administering to an individual a compound or salt thereof, wherein R₂ is aralkyl substituted with a substituent selected from the group consisting of unsubstituted alkyl and substituted phenoxy. In another aspect, the invention includes a method for the treatment or prevention of a substance use disorder comprising administering to an individual a compound or salt thereof, wherein R₂ is aralkyl substituted with methyl e.g.,

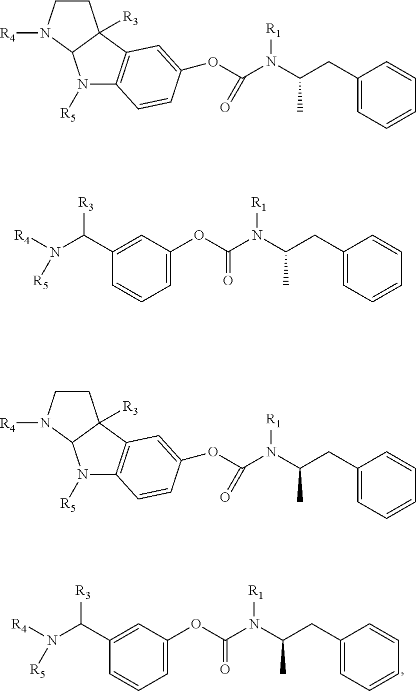

wherein R₁, R₃, R₄ and R₅ are as described herein.

In one aspect, the invention includes a method for the treatment or prevention of a substance use disorder comprising administering to an individual a compound or salt thereof, wherein R₂ is aralkyl substituted with a substituent selected from the group consisting of

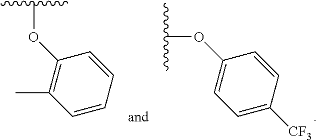

In one aspect, the invention includes a method for the treatment or prevention of a substance use disorder comprising administering to an individual a compound or salt thereof, wherein R₂ is substituted alkyl. In one aspect, the invention includes a method for the treatment or prevention of a substance use disorder comprising administering to an individual a compound or salt thereof, wherein R₂ is alkyl substituted with a substituent selected from the group consisting of unsubstituted alkyl, unsubstituted cycloalkyl, unsubstituted tricyclic ring, unsubstituted alkenyl-tricyclic ring, unsubstituted oxime and substituted oxime. In one aspect, the invention includes a method for the treatment or prevention of a substance use disorder comprising administering to an individual a compound or salt thereof, wherein R₂ is alkyl substituted with cyclohexyl. In another aspect, the invention includes a method for the treatment or prevention of a substance use disorder comprising administering to an individual a compound or salt thereof, wherein R₂ is alkyl substituted with

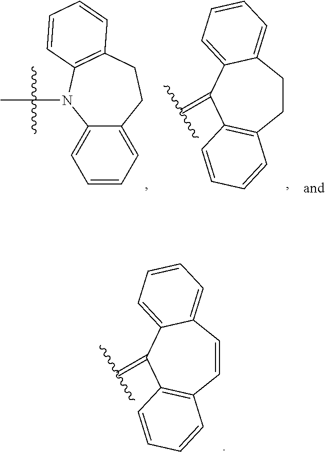

In another aspect, the invention includes a method for the treatment or prevention of a substance use disorder comprising administering to an individual a compound or salt thereof, wherein R₂ is alkyl substituted with

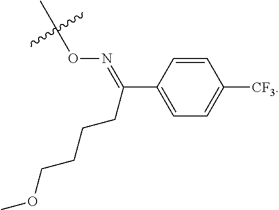

In one aspect, the invention includes a method for the treatment or prevention of a substance use disorder comprising administering to an individual a compound or salt thereof, wherein R₂ is substituted cycloalkyl. In one aspect, the invention includes a method for the treatment or prevention of a substance use disorder comprising administering to an individual a compound or salt thereof, wherein R₂ is cyclopropyl. In another aspect, the invention includes a method for the treatment or prevention of a substance use disorder comprising administering to an individual a compound or salt thereof, wherein R₂ is 1,2,3,4-tetrahydronaphthalene. In one aspect, the invention includes a method for the treatment or prevention of a substance use disorder comprising administering to an individual a compound or salt thereof, wherein R₂ is cycloalkyl substituted with aryl. In one aspect, the invention includes a method for the treatment or prevention of a substance use disorder comprising administering to an individual a compound or salt thereof, wherein R₂ is cycloalkyl substituted with a substituent selected from the group consisting of substituted phenyl and unsubstituted phenyl. In one aspect, the invention includes a method for the treatment or prevention of a substance use disorder comprising administering to an individual a compound or salt thereof, wherein R₂ is cycloalkyl substituted with phenyl wherein the phenyl is substituted with at least one halogen. In one aspect, the invention includes a method for the treatment or prevention of a substance use disorder comprising administering to an individual a compound or salt thereof, wherein R₂ is cycloalkyl substituted with phenyl which is substituted with at least one chlorine.

In another aspect, the invention includes a method for the treatment or prevention of a substance use disorder comprising administering to an individual a compound or salt thereof, wherein R₂ is substituted heteroaralkyl. In another aspect, the invention includes a method for the treatment or prevention of a substance use disorder comprising administering to an individual a compound or salt thereof, wherein R₂ is heteroaralkyl and the alkyl moiety is substituted with aryloxy. In another aspect, the invention includes a method for the treatment or prevention of a substance use disorder comprising administering to an individual a compound or salt thereof, wherein R₂ is heteroaralkyl and the alkyl moiety is substituted with

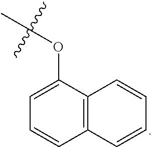

In one aspect, the invention includes a method for the treatment or prevention of a substance use disorder comprising administering to an individual a compound of Table 1 or a salt thereof.

TABLE 1
Exemplary Compounds of the Invention

| CMPD | | Compound | |
|---|---|---|---|
| No. | | Name | |
| 9 | 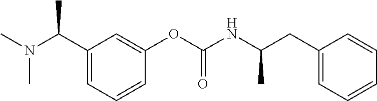 | S-riva-1- amphetamine | Sympathomimetic |
| 20 | 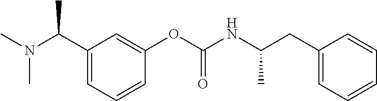 | S-riva-d- amphetamine | Sympathomimetic |
| 7 | 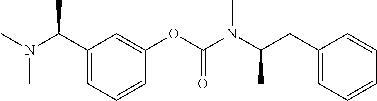 | S-riva-1- methamphetamine | Sympathomimetic |
| 13 | 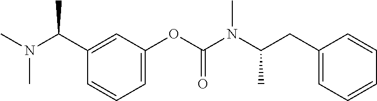 | S-riva-d- methamphetamine | Sympathomimetic |
| 14 | 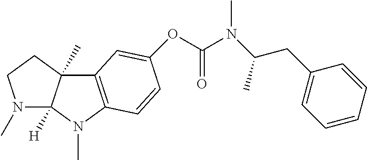 | Physo-d- amphetamine | Sympathomimetic |
| 15 | 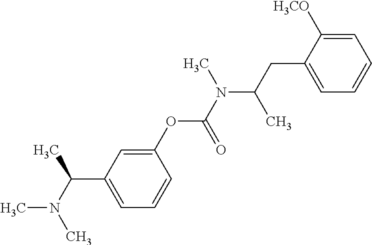 | S-riva- methoxyphenamine | Sympathomimetic |
| 16 | 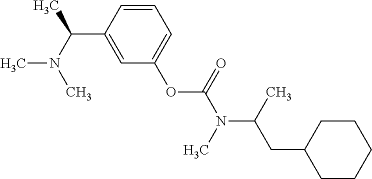 | S-riva- propylhexedrine | Sympathomimetic |
| 11 | 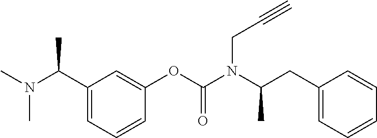 | S-riva- desmethylselegiline | Sympathomimetic |
| 17 | 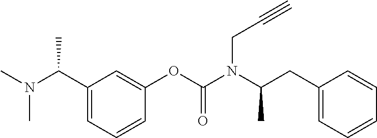 | R-riva- desmethylselegiline | Sympathomimetic |
| 18 | 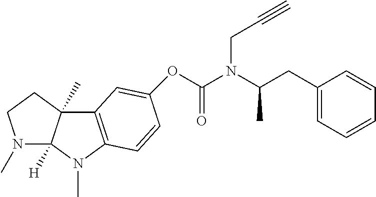 | Physo- desmethylselegiline | Sympathomimetic |
| 2 | 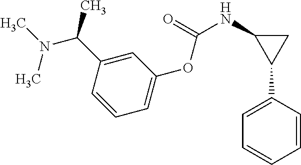 | S-riva- tranylcypromine | Sympathomimetic |
| 29 | 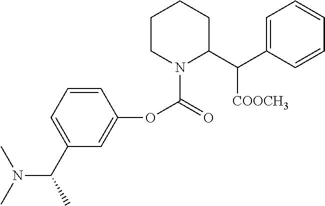 | S-riva-rac- methylphenidate | Sympathomimetic |
| 5 | 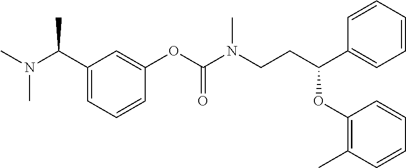 | S-riva- atomoxetine | Antidepressant |
| 3 | 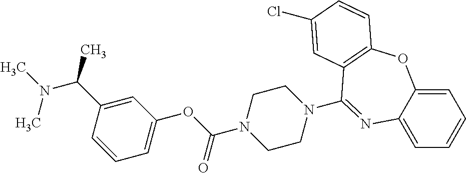 | S-riva- amoxapine | Antidepressant |
| 4A | 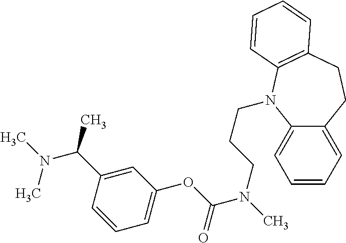 | S-riva- desipramine | Antidepressant |
| 5A | 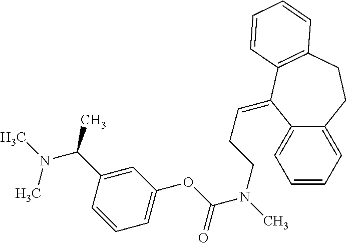 | S-riva- nortriptyline | Antidepressant |
| 6A | 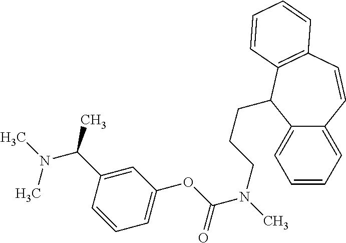 | S-riva- protriptyline | Antidepressant |
| 7A | 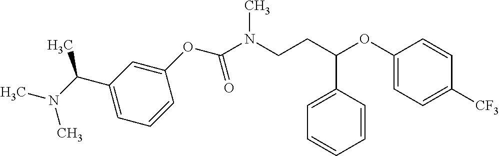 | S-riva- fluoxetine | Antidepressant |
| 8A | 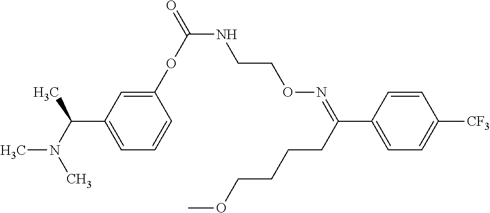 | S-riva- fluvoxamine | Antidepressant |
| 9A | 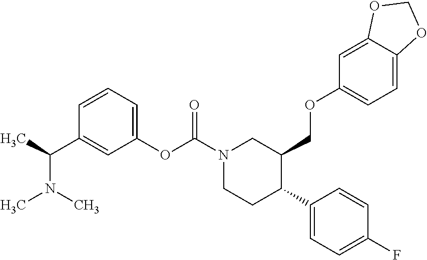 | S-riva- paroxetine | Antidepressant |
| 23 | 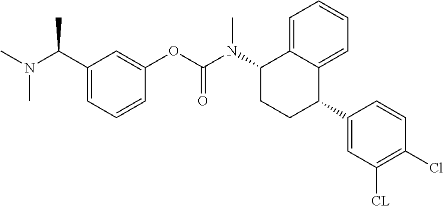 | S-riva-sertraline | Antidepressant |
| 10 | 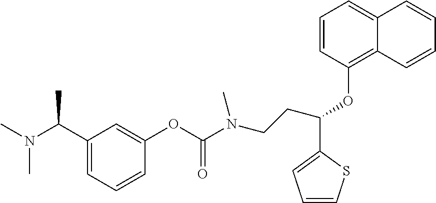 | S-riva- duloxetine | Antidepressant |
| 19 | 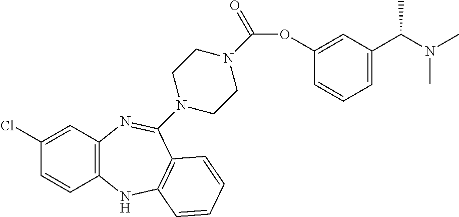 | S-riva- desmethyclozapine | Antipsychotic |
| 21 | 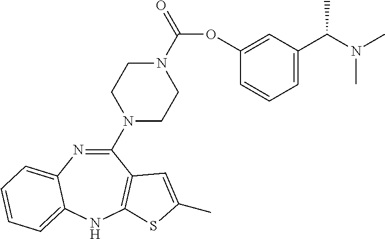 | S-riva- olanzapine | Antipsychotic |

In one aspect, the invention includes a method for the treatment or prevention of a substance use disorder comprising administering to an individual a compound having the formula:

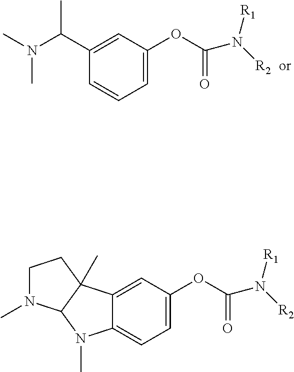

or a salt thereof, wherein R₁ and R₂ are as described above.

In another aspect, the invention includes a method for the treatment or prevention of a substance use disorder comprising administering to an individual a compound having the formula:

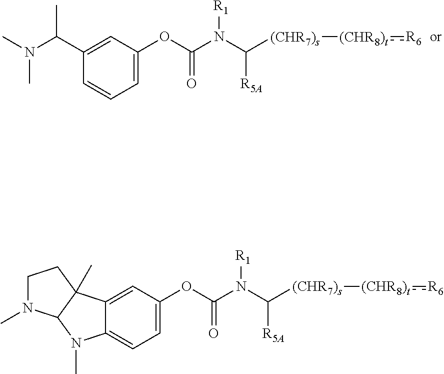

or a salt thereof, wherein R₁ is as described above. R_{5A} is selected from the group consisting of hydrogen, unsubstituted alkyl, and substituted alkyl. R₆ is selected from the group consisting of unsubstituted aryl, substituted aryl, unsubstituted cycloalkyl, substituted cycloalkyl, unsubstituted tricyclic ring, and substituted tricyclic ring. R₇ is selected from the group consisting of hydrogen, unsubstituted alkyl, and substituted alkyl. R₈ is selected from the group consisting of hydrogen, unsubstituted alkyl, substituted alkyl, substituted aryloxy, unsubstituted aryloxy. The variable s is 0 or 1. The variable t is 0 or 1. The variables s and t are not both 0. The dashed line “- - - ” is absent or taken together with the bond shown directly above it forms a double bond.

The invention includes a method for the treatment or prevention of a substance use disorder comprising administering to an individual a compound having the formula:

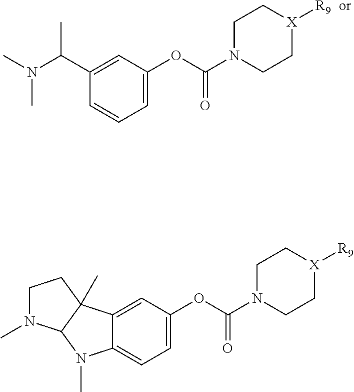

or a salt thereof. X is N or CH. R₉ is selected from the group consisting of hydrogen, substituted tricyclic ring, unsubstituted tricyclic ring, substituted aryl, unsubstituted aryl. The piperidine and piperazine ring is optionally substituted.

In one aspect, the invention includes a method for the treatment or prevention of a substance use disorder comprising administering a compound, wherein R₉ is a tricyclic ring selected from the group consisting of

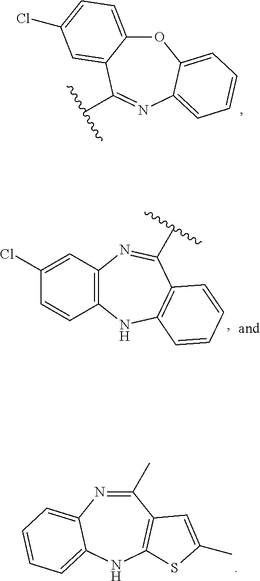

In one aspect, the invention includes a method for the treatment or prevention of a substance use disorder comprising administering a compound, wherein R9 is a tricyclic ring substituted with

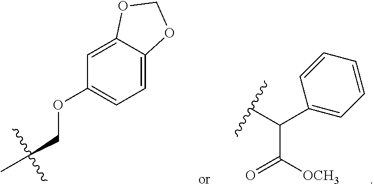

In one aspect, the invention includes a method for the treatment of prevention of a substance use disorder wherein the compound is a pharmaceutically acceptable salt thereof.

In one aspect, the invention includes a method for the treatment or prevention of a substance use disorder wherein the compound is administered as a pharmaceutical composition including a pharmaceutically acceptable carrier.

In one aspect, the invention includes a method for the treatment or prevention of a substance use disorder in an individual, comprising administering to an individual a compound of Table 1 or salt thereof or a pharmaceutical composition comprising a compound of Table 1 or salt thereof.

In one aspect, the invention includes a method for the treatment or prevention of a substance use disorder wherein the compound or salt thereof is an agonist medication. Agonist medications share pharmacological mechanisms of action with the abused substance and produce some effects in common with the abused substance. Agonist medications typically have a longer duration of action than an abused substance, and they are chronically administered under conditions that may produce tolerance to and/or prevent withdrawal from the abused drug.

In one aspect, the invention includes a method for the treatment or prevention of a substance abuse disorder, wherein one or more side effects selected from behavioral toxicity, insomnia, sleep disturbance, muscle twitching, cardiovascular responses (increased blood pressure and increased heart rate), thermoregulation problems, paranoia, hallucinations, dependence or pseudo-addiction are decreased or eliminated.

In one aspect, the invention includes a method for the treatment or prevention of a substance abuse disorder, wherein the substance abuse disorder is substance dependence or abuse with or without physiological dependence.

In one aspect, the invention includes a method for the treatment or prevention of a substance abuse disorder, wherein the substance associated with the disorder is selected from cocaine, amphetamine or amphetamine-like substance e.g., dextroamphetamine, nicotine, mu opioid agonists e.g., morphine, buprenorphine, fentanyl, levorphanol, meperidine, and methadone, and dextrorphan, and combinations of the above.

In one aspect, the invention includes a method for the treatment or prevention of a substance abuse disorder, wherein the substance use disorder is selected from drug withdrawal disorder, amphetamine withdrawal disorder, cocaine withdrawal, nicotine withdrawal, opioid withdrawal, and withdrawal symptoms due to addictive substances.

In one aspect, the invention includes a method for the treatment or prevention of a substance abuse disorder, wherein an effective amount of the compound or salt thereof is administered to treat the substance use disorder.

In one aspect, the invention includes a method for the treatment or prevention of a substance abuse disorder, wherein an effective amount of the compound or salt thereof is administered to prevent the substance use disorder.

In one aspect, the invention includes a method wherein the compound or salt thereof is administered to an individual in need of treatment thereof.

In one aspect, the invention includes a method for the treatment or prevention of a substance abuse disorder, wherein the compound or salt thereof is administered enterally, parenterally, orally or intramuscularly.

In one aspect, the invention includes a method for the treatment or prevention of a substance abuse disorder, wherein the compound or salt thereof is administered chronically. Chronic administration means administration over a long duration of time; continuing. In one aspect, the compound is administered over an 8-12 week period. In another aspect, the invention includes a method for the treatment or prevention of a substance abuse disorder, wherein the compound or salt thereof is administered subchronically.

In one aspect, the invention includes a method for the treatment or prevention of a substance abuse disorder, wherein the compound or salt thereof has a dose-limiting side effect. In one aspect, the invention includes a method for the treatment or prevention of a substance abuse disorder, wherein the side effect of the compound is nausea.

In one aspect, the invention includes a kit for carrying out the method of the invention as described herein.

In another aspect, the invention includes the use of compound or salt thereof, having a formula selected from

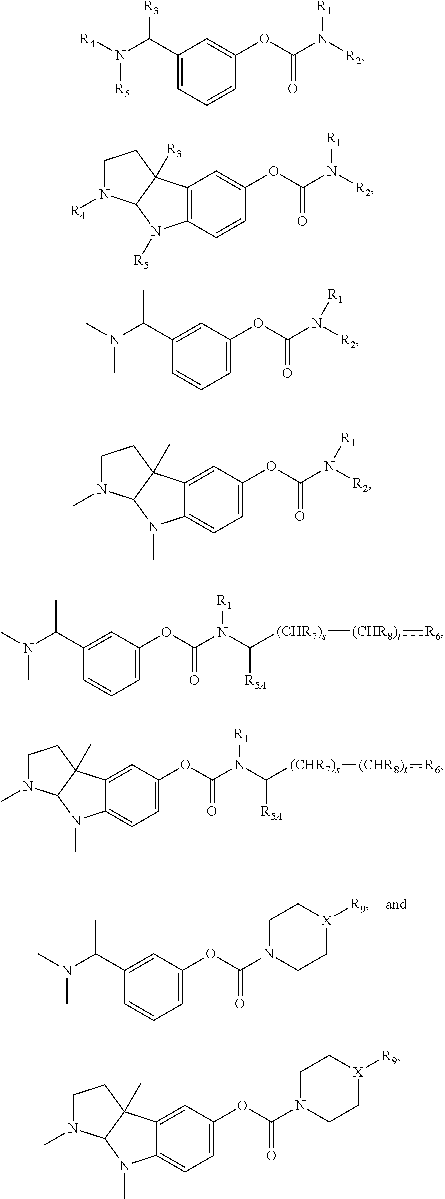

wherein R₁, R₂, R₃, R₄, and R₅, R_{5A}, R₆, R₇, R₈, s, t, - - - , X and R₉ are as describe manufacture of a medicament for the treatment or prevention of a substance use disorder. In one aspect, the invention includes the use of a compound of Table 1 or salt thereof, in the manufacture of a medicament for the treatment or prevention of a substance use disorder.

In one aspect, the invention includes a method for the treatment or prevention of a substance abuse disorder, wherein the compound or salt thereof inhibits acetylcholinesterase. In particular, a compound of the invention inhibits a cholinesterase by competing with a compound (e.g., acetylcholine (ACh) or butyrylcholine (BuCh)) that binds to the cholinesterase.

The cholinesterase is inhibited when it is prevented from inactivating a compound, such as the neurotransmitter ACh, to any degree that cholinesterase would act on the neurotransmitter in the absence of the carbamoyl ester. The carbamoyl ester binds to the cholinesterase to form a carbamoylated enzyme. Hydrolysis of the carbamoylated enzyme is much slower than that of, for example, an acetylated enzyme, which is formed by hydrolysis of its endogenous substrate acetylcholine. Inhibition of the cholinesterase by a carbamoyl ester molecule ceases when the carbamoylated enzyme is hydrolyzed. Upon hydrolysis of the carbamoylated enzyme, a released compound, such as an amine, becomes at least a component of a pharmacologically active agent.

Hydrolysis of the carbamoyl ester of the compounds of the invention, which thereby releases at least a component of a pharmacologically active agent, can be hydrolysis by an enzyme (e.g., a cholinesterase) or hydrolysis by other than an enzyme, such as by an acid (e.g., gastric acid).

The phrase “upon hydrolysis by reaction with an enzyme,” as used herein, refers to the two-step process of reaction of the carbamoyl ester with an enzyme to form a carbamoylated enzyme, and decomposition of the carbamoylated enzyme by reaction with H₂O.

Likewise, the phrase “upon hydrolysis by reaction with the cholinesterase,” as used herein, refers to the two-step process of reaction of the carbamoyl ester with the enzyme cholinesterase, to form a carbamoylated enzyme, and decomposition of the carbamoylated enzyme by reaction with H₂O.

The cholinesterase inhibited by the carbamoyl ester of the invention can be, for example, at least one member selected from the group consisting of an acetylcholinesterase (AChE) or a butyrylcholinesterase (BuChE). The carbamoyl ester can inhibit AChE alone, BuChE alone, or can inhibit both AChE and BuChE to similar or different degrees.

AChE is located on excitable membranes and inactivates ACh. The excitable membrane can be a presynaptic neuron or a postsynaptic neuron. AChE is also referred to as specific cholinesterase. BuChE is located on excitable membranes and non-neuronal tissue such as blood cells. BuChE is also referred to as pseudocholinesterase or nonspecific cholinesterase. AChE and BuChE are regulators of cholinergic neurotransmission in the central nervous system (brain and spinal cord), peripheral nervous system and autonomic nervous system (parasympathetic nervous system and sympathetic nervous system).

Upon hydrolysis of the carbamate bond of the carbamoylated enzyme, a released compound, such as a compound that includes an amine, becomes at least a component of a pharmacologically active agent. The term “becomes at least a component of a pharmacologically active agent,” as used herein, refers to the release of a compound, such as an amine-containing compound, as a consequence of hydrolysis of the carbamoylated enzyme. The compound released by hydrolysis of the carbamoylated enzyme is at least a portion of a pharmacologically active agent. In one embodiment, the compound released by the hydrolysis of the carbamoylated enzyme is a prodrug. The term “prodrug,” as used herein, refers to a compound, such as a carbamoyl ester of the invention, that is administered, but is not the actual drug desired in the treatment regimen and is transformed by metabolic processes to the actual drug desired in the treatment. The prodrug then can be modified to release a pharmacologically active agent. In another embodiment, the compound released by hydrolysis of the carbamoylated enzyme can, itself, be the pharmacologically active agent. Thus, a carbamoyl ester of the invention has a dual role as an inhibitor of a cholinesterase and as a delivery vehicle for a pharmacologically active agent.

Hydrolysis of the carbamoyl ester, resulting in the release of a pharmacologically active agent is shown by the schemes detailed below:

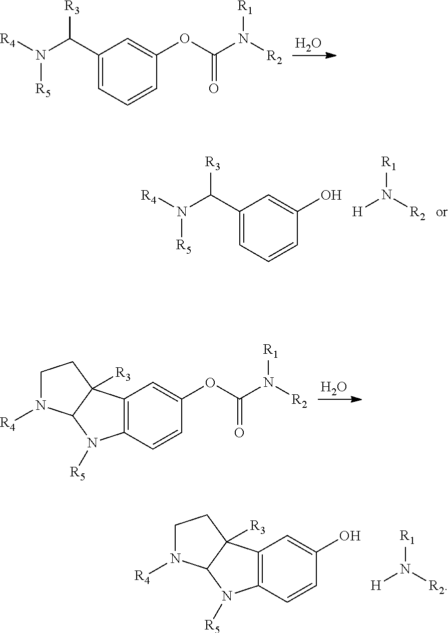

The term “pharmacologically active agent,” as used herein, refers to a compound that influences biological processes by altering the activity, localization and/or expression of molecules (e.g., neurotransmitters, peptides, proteins) which are directly or indirectly involved in the biological processes. For example, the pharmacologically active agent is a CNS active compound suitable for the treatment or prevention of a substance use disorder.

The term “alkyl,” used alone or as part of a larger moiety, includes both straight, branched, or cyclic saturated hydrocarbon chains containing one to twelve carbon atoms. The term “lower alkyl” means C₁₋₆ alkyl and is intended to include C₁, C₂, C₃, C₄, C₅, and C₆ alkyl groups.

A heteroalkyl, as used herein, is an alkyl group in which one or more carbon atoms is replaced by a heteroatom.

The term “aryl,” used alone or as part of a larger moiety as in “aralkyl” or “aralkoxy,” are carbocyclic aromatic ring systems (e.g. phenyl), fused polycyclic aromatic ring systems (e.g., naphthyl and anthracenyl) and aromatic ring systems fused to carbocyclic non-aromatic ring systems (e.g., 1,2,3,4-tetrahydronaphthyl and indanyl) having five to about fourteen carbon atoms.

The term “heteroaryl,” used alone or as part of a larger moiety as in “heteroaralkyl” or “heteroarylalkoxy,” refers to aromatic ring system having five to fourteen members and having at least one heteroatom. Preferably a heteroaryl has from one to about four heteroatoms. Preferred heteroalkyls are those wherein the heteroatom is selected from the groups consisting of oxygen, sulfur, nitrogen, phosphorase and halides. Examples of heteroaryl rings include pyrazolyl, furanyl, imidazolyl, isoxazolyl, oxadiazolyl, oxazolyl, pyrrolyl, pyridyl, pyrimidinyl, purinyl, pyridazinyl, pyrazinyl, thiazolyl, thiadiazolyl, isothiazolyl, triazolyl, thienyl, 4,6-dihydro-thieno[3,4-c]pyrazolyl, 5,5-dioxide-4,6-dihydrothieno[3,4-c]pyrazolyl, thianaphthenyl, 1,4,5,6,-tetrahydrocyclopentapyrazolyl, carbazolyl, benzimidazolyl, benzothienyl, benzofuranyl, indolyl, azaindolyl, indazolyl, quinolinyl, benzotriazolyl, benzothiazolyl, benzothiadiazolyl, benzooxazolyl, benzimidazolyl, isoquinolinyl, isoindolyl, acridinyl, and benzoisazolyl. Preferred heteroaryl groups are pyrazolyl, furanyl, pyridyl, quinolinyl, indolyl and imidazolyl.

An aralkyl group, as used herein, is an aryl substituent that is linked to a compound by a straight chain or branched alkyl group having from 1, 2, 3, 4, 5, 6, 7, 8, 9, 10, 11, to 12 carbon atoms. In one embodiment, the straight chain or branched alkyl group of the aralkyl group has from one to three carbon atoms. In one embodiment, the straight chain or branched alkyl group of the aralkyl group has three carbon atoms. In one embodiment, the alkyl group of the aralkyl group is a branched alkyl group having three carbon atoms. In one embodiment, the aralkyl group is

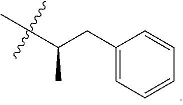

An heterocycloalkyl group, as used herein, is a heterocycle substituent that is linked to a compound by a straight chain or branched alkyl group having from one to twelve carbon atoms.

An heteroaralkyl group, as used herein, is a heteroaryl substituent that is linked to a compound by a straight chain or branched alkyl group having from one to twelve carbon atoms.

An aryl (including aralkyl, aralkoxy and the like) or heteroaryl (including heteroaralkyl and heteroaralkoxy and the like) may contain one or more substituents. Examples of suitable substituents include aliphatic groups, aryl groups, haloalkoxy groups, heteroaryl groups, halo and hydroxy.

The term “substituted,” as used herein, means that any one or more hydrogens on the designated atom is replaced with a selection from the indicated group, provided that the designated atom's normal valency is not exceeded, and that the substitution results in a stable compound.

In one embodiment, the compound of the invention includes an isomer or stereoisomer (e.g., d, l, dl, R, S, or RS). In all structures shown herein, it is to be understood that, whether a compound is represented as (+, −), dl (DL) or (R)(S), the invention is intended to include racemic mixtures, or pure compositions of one form of the compound, e.g. “d” or “l,” “R” or “S,” unless otherwise specified.

Methods to prepare the compounds of the invention are within the knowledge of one skilled in the art (see, for example, U.S. Pat. Nos. 5,665,880; 5,677,457; and WO 97/14694, the teachings of which are hereby incorporated by reference in their entirety).

The term “amphetamine,” such as is used when referring to “l-amphetamine” and “d-amphetamine,” means a compound represented by Formula G, including prodrugs and other structural and functional derivatives thereof wherein the primary amine group is available for substitution. In one embodiment, the amphetamine is the compound represented by Formula G:

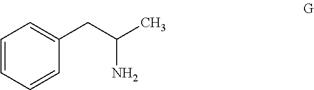

The dextro enantiomer of amphetamine is referred to as the d, (+), D or S isomer and is represented by the following structural formula:

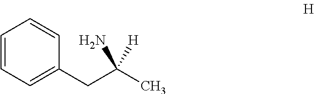

The levo enantiomer of amphetamine can be referred to as the l, (−), L or R and is represented by the following structural formula:

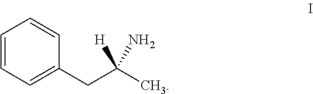

Racemic mixtures of d-amphetamine and l-amphetamine are referred to as dl, (+,−), DL or (R)(S).

An (R)-(−)-amphetamine employed in the methods of the invention is represented by the structural formula:

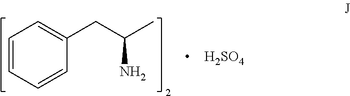

Formula J is also referred to as levo-amphetamine sulfate or l-amphetamine sulfate. Formula J has the molecular formula C₁₈H₂₈N₂O₄S and a molecular weight of 368.50. The IUPAC chemical name of Formula J is (−)-1-methyl-2-phenylethylamine sulfate (2:1) and the CAS chemical name (−)-α-methylphenethylamine sulfate (2:1).

The term “methamphetamine,” such as is used when referring to “l-methamphetamine” and “d-methamphetamine,” means a compound represented by Formula K:

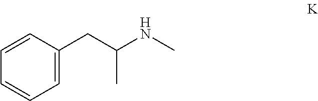

The (R)-(−)-methamphetamine can be represented by the structural formula:

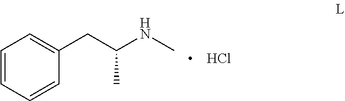

Formula L is also referred to levo-methamphetamine HCl, 1-methamphetamine HCl or levomethamphetamine HCl. Formula L has the molecular formula C₁₀H₁₆NCl.

In still another embodiment, the (R)-(−)-methamphetamine can be represented by the structural formula:

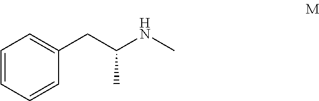

Formula M is also referred to levo-methamphetamine, levo-desoxyephedrine, 1-desoxyephedrine or levmetamfetamine.

An “agent,” as used herein, refers to a compound that can produce a physical, chemical or biological effect that can be stimulatory (e.g., an activating agent) or inhibitory (e.g., a blocking agent). Agents that are stimulatory can be agonists. Agents that are inhibitory can be antagonists or inverse agonists. Inverse agonists are compounds or molecules that down-regulate receptor activated activity thereby acting in a manner that is the opposite of an agonist to the receptor. Thus, exposure or administration of an inverse agonist can result in a diminished response compared to exposure or administration of an agonist.

A “modulator,” as used herein, refers to a compound that regulates, adjusts or adapts a biological pathway or receptor-mediated signal transduction pathway. The modulators can stimulate or inhibit a biological pathway or receptor-mediated signal transduction pathway.

For example, an adenosine receptor modulator can increase the capacity of adenosine to bind the receptor, decrease the capacity of adenosine to bind the receptor, directly bind to the receptor (e.g., an agonist or inverse agonist) and have an effect or otherwise interact with the receptor to regulate, adjust or adapt a biological pathway associated with an adenosine receptor mediated signal transduction pathway.

The carbamoyl ester of the invention can inhibit cholinesterase activity, which can be expressed as an IC₅₀. The term “IC₅₀,” as used herein, refers to the concentration of a drug, compound, molecule or carbamoyl ester that inhibits an activity or effect by 50%, by reducing binding of a competitor molecule to a protein (e.g., a receptor) by 50%; or by reducing the level of an activity (e.g., cholinesterase activity) by 50%.

As used herein, an “individual” is any mammal. A mammal can be a rodent (such as a rat, mouse or guinea pig), domesticated animal (such as a dog or cat), ruminant animal (such as a horse or a cow) or a primate (such as a monkey or a human). In a preferred embodiment, the individual is a human.

The terms “subchronic” means of intermediate duration.

The term “substance use disorder” means a complex behavioral disorder characterized by preoccupation with obtaining alcohol or other drugs (AOD) and a narrowing of the behavioral repertoire towards excessive consumption and loss of control over consumption. It may also be accompanied by the development of tolerance and withdrawal and impairment in social and occupational functioning. Synonyms for substance use disorder are alcohol abuse, other drug problem, alcoholism, other drug dependence, addiction etc.

The term “compounds of the invention” refers to the carbamoyl esters used in the methods of the invention and described herein.

The carbamoyl esters of the invention can be employed in the methods, pharmaceutical compositions, kits and assays of the invention in a single dose or in multiple doses. The multiple doses can be administered as multiple doses in a single day, as a single daily dose administered for more than one day, as multiple doses administered daily for more than one day, or as a single dose on any given day followed or preceded by multiple doses in the intervening days. The multiple doses can be administered for a day, days, a week, weeks, a month, months, a year or years.

The carbamoyl esters of the invention can be administered in the methods of the invention to an individual acutely (briefly or short-term) or chronically (prolonged or long-term). For example, the carbamoyl esters of the invention can be used in methods to treat an individual by administering the carbamoyl ester to the individual once a day, multiple times (e.g., 2, 3, 4) in a day, for a day, days, a week, weeks, a month, months or years.

In one embodiment, the dose of the carbamoyl ester can be about 0.1 mg, about 1 mg, about 2.5 mg, about 5 mg, about 10 mg, about 15 mg, about 20 mg, about 25 mg, about 40 mg, about 50 mg, about 75 mg, about 90 mg, about 100 mg, about 150 mg, about 200 mg, about 250 mg, about 500 mg, about 750 mg or about 1000 mg.

In another embodiment, the dose of the carbamoyl ester can be between about 1 mg to about 100 mg; between about 2 mg to about 50 mg; or between about 5 mg to about 25 mg.

In still another embodiment, each dose of a multiple dose can be about 0.1 mg, about 1 mg, about 2.5 mg, about 5 mg, about 10 mg, about 20 mg, about 25 mg, about 40 mg, about 50 mg, about 75 mg, about 90 mg, about 100 mg, about 150 mg, about 200 mg, about 250 mg, about 500 mg, about 750 mg or about 1000 mg.

In a further embodiment, each dose of a multiple dose can be between about 1 mg to about 100 mg; between about 2 mg to about 50 mg; or between about 5 mg to about 25 mg.

The carbamoyl ester and the pharmacologically active agent are administered in the methods of the invention or employed in the assays and kits of the invention in an effective amount. The term “effective amount,” “amount effective,” or “therapeutically effective amount,” when referring to the amount of the carbamoyl ester or pharmacologically active agent, is defined as that amount, or dose, of the carbamoyl ester or pharmacologically active agent that is sufficient for therapeutic efficacy.

The carbamoyl ester can optionally be used in the methods, kits and assays of the invention with an acceptable carrier. The selection of an acceptable carrier will depend upon the method, kit or assay. For example, an acceptable carrier in an in vitro method, assay or kit can be saline, a suitable buffer or cell culture media.

The carbamoyl esters of the invention can be administered alone or as admixtures with conventional excipients, for example, pharmaceutically, or physiologically, acceptable organic, or inorganic carrier substances suitable for enteral or parenteral application which do not deleteriously react with the compound employed in the method. Suitable pharmaceutically acceptable carriers include water, salt solutions (such as Ringer's solution), alcohols, oils, gelatins and carbohydrates such as lactose, amylose or starch, fatty acid esters, hydroxymethycellulose, and polyvinyl pyrolidine. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances which do not deleteriously react with the compounds employed in the methods of the invention. The preparations can also be combined, when desired, with other active substances to reduce metabolic degradation.

Preferred methods of administration of the carbamoyl esters are oral administration (such as a tablet or capsule). The carbamoyl ester alone, or when combined with an admixture, can be administered in a single or in more than one dose over a period of time to confer the desired effect.

The carbamoyl esters can be administered to a target site in an individual. The target site selected can depend on the disorder to be treated.

When parenteral application is needed or desired, particularly suitable admixtures for the carbamoyl esters are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampules are convenient unit dosages. The carbamoyl esters employed in the methods, assays or kits of the invention can also be incorporated into liposomes or administered by transdermal pumps or patches. Pharmaceutical admixtures suitable for use in the present invention are well-known to those of skill in the art and are described, for example, in Pharmaceutical Sciences (17th Ed., Mack Pub. Co., Easton, Pa.) and WO 96/05309, the teachings of which are hereby incorporated by reference.

The dosage and frequency (single or multiple doses) administered to an individual can vary depending upon a variety of factors, including, for example, the disorder to be treated, the duration of the disorder, the pharmacologically active agent to be delivered or cognition; size, age, sex, health, body weight, body mass index and diet of the individual; nature and extent of symptoms of the disorder, kind of concurrent treatment, complications from the disorder or other health-related problems of the human being treated.

Other therapeutic regimens or agents can be used in conjunction with the methods and carbamoyl esters employed in the methods of the invention. Adjustment and manipulation of established dosages (e.g., frequency and duration) are well within the ability of those skilled in the art.

The present invention is further illustrated by the following examples, which are not intended to be limiting in any way.

### EXEMPLIFICATION

### Example 1

### Synthesis of S-riva-atomoxetine (5)

(S)-(−)-3′-hydroxyphenylethyldimethylamine (96 mg, 0.58 mmol) (1) was dissolved in 4 ml of dry ethyl acetate. N,N′-carbonyldiimidazole powder (283 mg , 1.74 mmol) was added and the mixture stirred at room temperature for 20 h. Acetic acid (313 mg, 5.22 mmol) was then added to the mixture, followed by the addition of 162 mg (−)-atomoxetine (4, 0.63 mmol). The resulting mixture was stirred at room temperature overnight. Saturated sodium bicarbonate solution was added to the mixture and the aqueous and organic layers separated. The aqueous layer was extracted twice with ethyl acetate. The organic layers were combined, dried over NaHCO3, evaporated and purified with a silica gel column (eluted with 25% ethyl acetate in hexane with 1% triethylamine) to yield 101 mg of the carbamoyl ester (5) (0.23 mmol, 39.0% yield).

The carbamoyl ester (5) was confirmed by NMR. ¹H-NMR of the HCl salt (CDCl₃, 400 MHz): δ 1.808 and 1.825 (d, 3H, J=6.8 Hz, CH₃), 2.090-2.320 (m, 2H), 2.262 (ma) and 2.325 (mi) (s, 3H, CH₃), 2.506-2.541 (m, 3H, CH3), 2.658-2.698 (m, 3H, CH3), 3.002 (ma) and 3.082 (mi) (s, 3H, CH3), 3.520-3.575 (m, 1H, CH), 3.662-3.700 and 3.892-3.961 (m, 1H, CH), 4.048-4.123 (m, 1H, CH), 5.180-5.252 (m, 1H, CH), 6.535-6.582 (m, 1H, CH arom.), 6.729-6.787 and 6.902-6.957 (m, 3H, 3×CH arom.), 7.007-7.086 (m, 2H, 2×CH arom.), 7.224-7.428 (m, 7H, 7×CH arom.), 12.620 (bs, 1H, HCl).

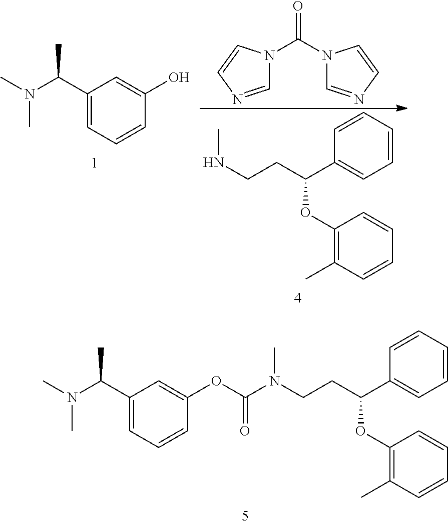

Free base 5 was converted into the hydrochloride salt following the procedure described below:

The carbamoyl ester (5) was dissolved in chloroform (3 mL per mmol free base 5). A solution of 1M HCl in ether (1.5-2 molar equivalents) was added dropwise at 0° C. Upon completion of addition of hydrochloric acid, the mixture was allowed to warm to room temperature. Solvents were removed by evaporation and the residue dried under vacuum to yield the hydrochloride salt of the carbamoyl ester (5) visible as a white to off-white solid.

### Example 2

### Synthesis of S-riva-l-methamphetamine (7)

4-nitrophenychloroformate powder (0.179 g, 0.86 mmol) was added to a solution of 0.12 g (0.72 mmol) (−)-3′-hydroxyphenylethyldimethylamine (1) and 0.22g (2.17 mmol) triethylamine in 10 ml of dry dichloromethane (0.86 mmol) at 0° C. The solution was stirred at 0° C. for 5 min followed by stirring at room temperature for an additional 30 minutes. A solution of 0.107 g 1-methamphetamine (6) in 2 ml of dry dichloromethane was then added, and the resulting solution stirred at room temperature for 2 hours. The solvent was evaporated and the residue applied to a silica gel column. The compound (7) was eluted with 3% acetone in ethyl acetate containing 1% triethylamine. Fractions containing compound (7) were combined and concentrated to yield 0.15 g of the compound (7) (0.44 mmol, 61% yield).

The compound (7) was confirmed by NMR. ¹H-NMR (CDCl3, 300 MHz): δ 1.192 (mi) and 1.275 (ma) (d, 3H, J=6.8 Hz, CH₃), 1.305 and 1.326 (d, 3H, J=3.0 Hz, CH₃), 2.162 and 2.167 (s, 6H, 2×CH₃), 2.746 (dd, 1H, J=13.7 and 6.8 Hz, CHH), 2.850 (dd, 1H, J=13.7 and 6.8 Hz, CHH), 2.868 and 2.886(s, 3H, CH₃), 3.165-3.217 (m, 1H, CH), 4.558-4.633 (m, 1H, CH), 6.665 and 6.855 (bd, 1H, J=7.9 Hz, CH arom.), 6.723 and 6.928 (bs, 1H, CH arom.), 7.065 (bd, 1H, J=7.2 Hz, CH arom.), 7.176-7.305 (m, 6H, CH arom.).

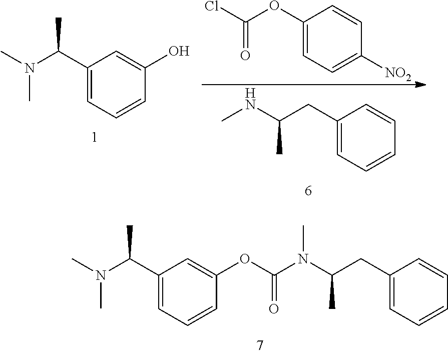

### Example 3A

### Synthesis of S-riva-l-amphetamine (9)

At room temperature, diisopropylethylamine (5.16 g, 40 mmol) and CDI powder (6.48 g, 40 mmol) were added to a suspension of 7.34 g of 1-amphetamine sulfate (8) (40 mmol) in 140 ml of dichloromethane. The resulting mixture was stirred at room temperature for 1 h. (−)-α-3′-hydroxyphenylethyldimethylamine (1) (3.3 g, 20 mmol), which had been mixed with 0.8 g sodium hydride (60% dispersion in mineral oil) in dry toluene (120 ml) for 30 minutes, was added to the mixture and the dichloromethane removed under reduced pressure. The resulting suspension was heated to 85° C. overnight with stirring. The reaction mixture was extracted with 0.5 M HCl (200 ml). The aqueous layer was washed with ethyl acetate, basified at 0° C. to pH ˜11 with sodium bicarbonate and 0.5 N NaOH and extracted with ethyl acetate (3×100 ml). The organic layers were combined, dried over sodium sulfate and evaporated. The residue was purified with a silica gel column. Elution with a mixture of 20-30% ethyl acetate with 1% triethylamine in hexane yielded 1.53 g of the carbamoyl ester (9) (4.7 mmol, 23.5% yield).

The carbamoyl ester (9) was confirmed by NMR. ¹H-NMR (CDCl3, 300 MHz): δ 1.179 (d, 3H, J=6.6 Hz, CH₃), 1.331 (d, 3H, J=6.7 Hz, CH₃), 2.174 (s, 6H, 2×CH₃), 2.789 (dd, 1H, J=13.4 and 7.2 Hz, CHH), 2.832 (dd, 1H, J=13.4 and 5.9 Hz, CHH), 3.228 (q, 1H, J=6.7 Hz, CH), 3.980-4.062 (m, 1H, CH), 4.856 (bd, 1H, J=7.2 Hz, NH), 6.955 (bd, 1H, J=7.4 Hz, CH arom.), 7.018(bs, 1H, CH arom.), 7.095 (bd, 1H, J=7.7 Hz, CH arom.) 7.186-7.303 (m, 6H, CH arom.).

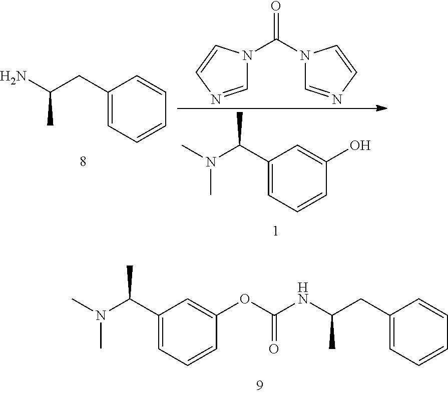

### Example 3B

### Alternative Synthesis of S-riva-L-amphetamine (9)

(S)-(−)-3′-hydroxyphenylethyldimethylamine (1) (1.2 g, 7.3 mmol) was dissolved in 20 ml of dry ethyl acetate. N,N′-carbonyldiimidazole powder (2.37 g, 14.6 mmol) was added and the mixture stirred at 85° C. overnight. After cooling to 0° C., 3.3 g of acetic acid (55.0 mmol) was added, followed by the addition of 2.8 g of 1-amphetamine (8) (20.7 mmol). The mixture was stirred at room temperature for 36 h. Water (20 ml) and 1M HCl (20 ml) were added and the aqueous and organic layers separated. The organic layer was extracted with 0.5M HCl. The aqueous layers were combined and washed with ether twice, basified with NaHCO₃ and 0.5 N NaOH to pH ˜11 and extracted with ether. The ether layer was dried over NaHCO₃, evaporated and purified with silica gel chromatography. Elution with a mixture of 25% ethyl acetate with 1% triethylamine in hexane yielded 0.93 g of the carbamoyl ester (9) (2.85 mmol, 39% yield).

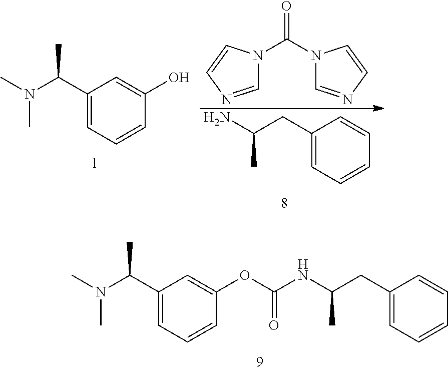

### Example 4

### Synthesis of S-riva-desmethylselegiline (11)

Triphosgene (85.5 mg, 0.28 mmol) was dissolved in 2 ml of dry dichloromethane.

To this solution, a mixture of 145 mg of desmethylselegiline (10) (0.84 mmol) and 110 mg of diisopropylethylamine (DIEA) (0.85 mol) in 1 ml of dry dichloromethane was added at 0° C. and allowed to react for 10 minutes. The mixture was stirred at room temperature for 60 hours, and subsequently added to a suspension of (−)-_-3′-hydroxyphenylethyldimethylamine (1) (92 mg, 0.55 mmol) and sodium hydride (68 mg, 60% dispersion in mineral oil) in dry acetonitrile, which had been stirred at room temperature for 1 hour. The resulting mixture was stirred at room temperature overnight. The solvents of the above mixture were removed under reduced pressure. The residue was dissolved in 0.5 M HCl and washed with ether. The aqueous layer was basified with sodium bicarbonate and extracted with ethyl acetate (3×20 ml). The organic layer was washed with 0.5 N NaOH (200 ml), dried over sodium sulfate and evaporated. The residue was purified with a silica gel column (eluted with 30-60% ethyl acetate in hexane with 1% triethylamine) to yield 185 mg of the carbamoyl ester (11) (0.508 mmol, 92.3% yield).

The carbamoyl ester (11) was confirmed by NMR. ¹H-NMR (CDCl3, 300 MHz): δ 1.339 (d, 3H, J=6.6 Hz, CH₃), 1.327-1.415 (m, 3H, CH₃), 2.187 (s, 6H, 2×CH₃), 2.215-2.258 (m, 1H, CH), 2.843-2.870 (m, 1H, CH), 3.063 (dd, 1H, J=13.5 and 7.5 Hz, CHH), 3.230 (q, 1H, J=6.6 Hz, CH), 4.043-4.118 (m, 2H, 2×CH), 4.372-4.411 (m, 1H, CH), 6.846-7.024 (m, 2H, 2×CH arom.), 7.108 (bd, 1H, J=7.7 Hz, CH arom.), 7.202-7.313 (m, 6H, CH arom.).

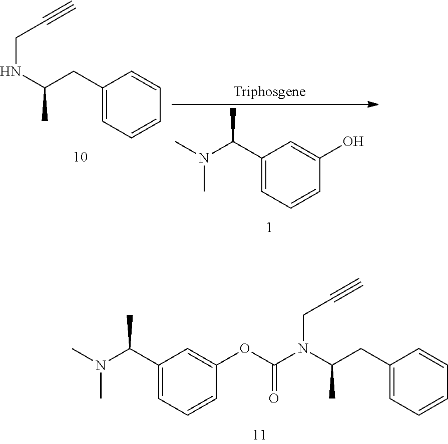

### Example 5

### Synthesis of S-riva-d-amphetamine (20)

(S)-(−)-3′-hydroxyphenylethyldimethylamine (1) (81 mg, 0.49 mmol) was dissolved in 4 ml of dry ethyl acetate. N,N′-carbonyldiimidazole powder (199 mg, 1.23 mmol) was added and the mixture was stirred at room temperature for 20 h. Acetic acid (184 mg, 3.07 mmol) was added, followed by the addition of 186 mg of d-amphetamine (19) acetate salt (0.96 mmol). The mixture was stirred at room temperature overnight. Water (5 ml) and 1M HCl (5 ml) were added and the aqueous and organic layers separated. The organic layer was extracted with 0.5M HCl. The aqueous layers were combined, washed with ether twice and basified with NaHCO₃ and 0.5 N NaOH to pH ˜11, followed by extraction with ether. The ether layer was dried over NaHCO₃, evaporated and purified with a silica gel column (eluted with 25% ethyl acetate in hexane with 1% triethylamine) to yield 95 mg of carbamoyl ester (20) (0.29 mmol, 59.4% yield).

The carbamoyl ester (20) was confirmed by NMR. ¹H-NMR (CDCl3, 300 MHz): δ 1.192 (d, 3H, J=6.6 Hz, CH₃), 1.367 (d, 3H, J=6.7 Hz, CH₃), 2.205 (s, 6H, 2×CH₃), 2.759 (dd, 1H, J=13.4 and 7.2 Hz, CHH), 2.896 (dd, 1H, J=13.4 and 5.9 Hz, CHH), 3.295 (q, 1H, J=6.6 Hz, CH), 3.990-4.044 (m, 1H, CH), 4.847 (bd, 1H, J=7.2 Hz, NH), 6.966 (bd, 1H, J=7.4 Hz, CH arom.), 6.976 (bs, 1H, CH arom.), 7.114 (bd, 1H, J=7.7 Hz, CH arom.) 7.191-7.324 (m, 6H, CH arom.).

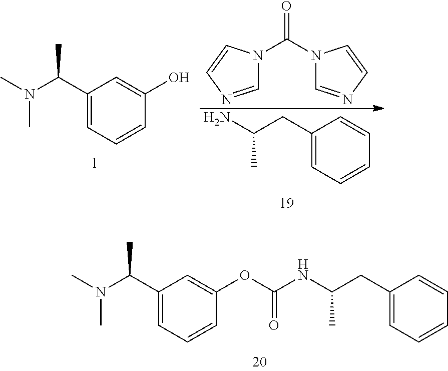

### Example 6

### Compound Synthesis

Compounds of the invention are produced by coupling of Rₐ-phenol and Q-H using methods known to those skilled in the art. For example,

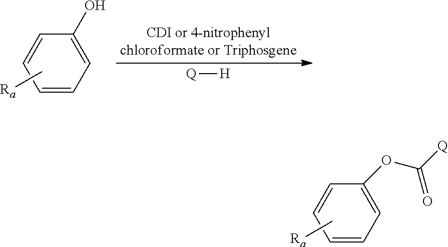

wherein Rₐ represents the appropriate phenyl substituents for a stigmine, such as rivastigmine or physostigmine, and Q represents an amine-containing pharmacologically active agent. For example,

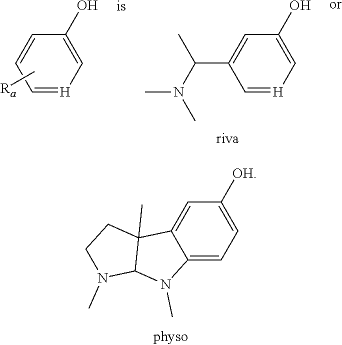

Exemplary compounds are shown in Table A.

TABLE A

| Starting material | Reagents/conditions | Results |
|---|---|---|
| Desipramine (300 | Desipramine is treated with sodium | Compound 4A (240 mg, 52% |
| mg, 1.0 mmol) | bicarbonate and riva carbamate | yield, >95% by HPLC.) |
| | imidazole solution (2.0 mmol, 2.0 | isolated by column |
| | eq.) in dichloromethane (8 mL). | chromatography. |
| Fluvoxamine | Fluvoxamine is treated with sodium | Compound 8A (10 mg, 8% |
| maleate (100 mg, | bicarbonate and riva carbamate | yield, 90% purity by HPLC) |
| 0.23 mmol) | imidazole solution (0.66 mmol, 3.0 | isolated by preparative TLC. |
| | eq.) in dichloromethane (7 mL). | |
| Fluoxetine | Fluoxetine is treated with | Compound 7A isolated by |
| hydrochloride (100 | diisopropylethylamine (0.63 mmol, | preparative TLC to give 30 mg, |
| mg, 0.29 mmol) | 2.2 eq.) and riva carbamate | 20% yield, 80% purity by |
| | imidazole solution (0.63 mmol, 2.2 | HPLC. |
| | eq) in dichloromethane (6 mL). | |
| Paroxetine (87 mg, | Riva carbamate soln in | Compound 9A (49 mg, 83% |
| 0.26 mmol) | dichloromethane (S-rivastigmine | purity). |
| | coupled with carbonyldiimidazole) | |
| | 1.2 mmol., dichloromethane (4 mL) | |
| Sertraline maleate | Sertraline is treated with sodium | Compound 23 |
| (250 mg, 0.73 | bicarbonate and riva carbamate | |
| mmol) | imidazole solution (1.5 mmol, 2.05 | |
| | eq.) in the presence of | |
| | diisopropylethylamine (2.87 mmol, | |
| | 3.9 eq.) in dichloromethane (15 mL). | |
| Methylphenidate | 1)Methylphenidate is treated with | Compound 29 |
| HCl (270 mg, 1.0 | 2.0M aq. soln. of Na₂CO₃, dried and | |
| mmol) | concentrated, riva carbamate soln | |
| | (2.4 mL of 0.25M soln in | |
| | dichloromethane), dichloromethane | |
| | (2 mL) | |
| | 2)diisopropylethylamine (130 mg, | |
| | 1.0 mmol) added and stirred | |
| Protriptyline HCl (2 | Carbonyldiimidazole (6.67 mmol), | Compound 6A |
| g, 6.67 mmol) | (S)-rivastigmine phenol (6.67 | Purified twice on silica column |
| | mmol), diisopropylethylamine (10.0 | chromatography to give 1.15 g |
| | mmol), dichloromethane (60 mL) | of the desired product (HPLC |
| | | purity > 99%). |
| Fluoxetine HCl (2 | Carbonyldiimidazole (6 mmol), (S)- | Compound 7A |
| g, 6 mmol) | rivastigmine phenol (6 mmol), | Purified on a silica column |
| | diisopropylethylamine (9 mmol), | chromatography to give 1.05 g |
| | dichloromethane (40 mL) | of the desired product (HPLC |
| | | purity > 99%) |
| Duloxetine (740 | Carbonyldiimidazole (2.6 mmol), | Compound 10A |
| mg, 2.5 mmol) | (S)-rivastigmine phenol (2.7 mmol), | |
| | dichloromethane (10 mL) | |
| Fluvoxamine | Carbonyldiimidazole (1.05 mmol), | Compound 8A |
| maleate (434 mg, 1 | (S)-rivastigmine phenol (1.1 mmol), | LC/MS of the reaction mixture |
| mmol) | diisopropylethylamine (3 mmol), | showed the mass of the |
| | dichloromethane (6 mL) | product. |
| Fluvoxamine | Carbonyldiimidazole (6.05 mmol), | Compound 8A |
| maleate (2.5 g, 5.7 | (S)-rivastigmine phenol (6.3 mmol), | LC/MS of the reaction mixture |
| mmol) | diisopropylethylamine (17.3 mmol), | showed the mass of the |
| | dichloromethane (40 mL) | product. |

### Example 7

### Purification of Compounds of the Invention from S-rivastigmine and l-amphetamine

Samples of the carbamoyl ester obtained from S-rivastigmine and 1-amphetamine were dissolved in water (30 mL) and adjusted to a pH of ˜10 using 2.0 M aq. solution of Na₂CO₃. The carbamoyl ester free base was then extracted with dichloromethane (2×30 mL), dried (Na₂SO₄) and concentrated using a rotovap. The residue was passed through a silica column using heptanes (74%), ethyl acetate (25%) and triethylamine (1%) as the solvent. The fractions were evaporated using a rotovap and dried under high vacuum overnight. The residue was taken up in water (6 mL), followed by the addition of 2.0 M HCl (3 mL) gave a clear homogeneous solution. It was then lyophilized to give the carbamoyl ester HCl (278 mg, HPLC purity >99%). The carbamoyl ester is shown below:

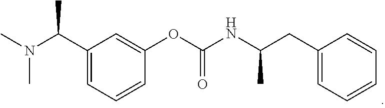

The lyophilized material was a white, free flowing powder where as the sample before purification and lyophilization was sticky and was hard to transfer.

### Examples 8A and 8B

### Preparation of Hydrochloride Salts of Compounds of the Invention

### Example 8A

A compound of the invention is dissolved in chloroform (3 ml per mmol compound). A solution of 1M HCl in ether (1.5-2 molar equivalents) is added dropwise at 0° C. Upon completion of addition of hydrochloric acid, the mixture is allowed to warm to room temperature. Solvents are removed by evaporation and the residue dried under vacuum to yield the hydrochloride salt of the compound.

### Example 8B

A compound is dissolved in water and adjusted to a pH of ˜10 using 2.0 M aq. solution of Na₂CO₃. The compound is then extracted with dichloromethane (2×30 mL), dried (Na₂SO₄) and concentrated. The residue is passed through a silica column using heptanes (74%), ethyl acetate (25%) and triethylamine (1%) as the solvent. The fractions are evaporated using a rotovap and dried under high vacuum overnight. The residue is taken up in water (6 mL), followed by the addition of 2.0 M HCl (3 mL). The solution is then lyophilized to give the compound as its HCl salt.

### Example 9

### Compounds Inhibit Acetylcholinesterase In Vitro

All reagents employed in these experiment were of analytical grade. Acetylthiocholine iodide and 5,5′-dithiobis-(2-nitro)benzoic acid (DTNB) and human recombinant acetylcholinesterase (C1682) were purchased from Sigma Chemical Co (St. Louis, Mo.).

Acetylcholinesterase activity of compounds was determined at 25° C. by a modification of the colorimetric method of Ellmann, et al. (Biochem. Pharmacol., 7:88-95 (1961)). The enzyme, compound or stigmine and buffer were preincubated for 30 minutes. At the end of the preincubation period, the substrate acetylthiocholine was added. The final assay mixture contained 10mM Tris-buffer (pH 8), 0.3 mM Acetylthiocholine and 0.33 mM DTNB and 0.08 U/ml enzyme. At least five (5) different concentrations of the compound or stigmine were assayed per 1050 experiment.

Hydrolysis of acetylthiocholine was monitored indirectly by measurement of the formation of the conjugate between thiocholine and DTNB. Optical density at 405 nm was recorded during 5 minutes employing a microplate spectrophotometer (Polarstar, BMG Labtech) and plotted against time. The inverse of the initial rates for a range of inhibitor concentrations was plotted against concentration (Dixon Plot) to give the 1050 value (the concentration at which enzyme activity is inhibited by 50%) as the opposite value of the x-intercept (Burlingham, et al., J. Chem. Ed., 80:214-218 (2003)).

The results are summarized as follows:

| | AChE | |
|---|---|---|
| Compound | IC₅₀ | |
| Rivastigmine | 2,615 | nM |
| 5 | 460 | nM |
| 7 | 302 | nM |
| 9 | 404 | nM |
| 13 | 5,440 | nM |
| 14 | 253 | nM |
| 20 | 449 | nM |

| | | | AChE |
|---|---|---|---|
| Cmpd | | | IC50 |
| # | Name | Compound Structure | (μM) |
| 1 | S-rivastigmine | 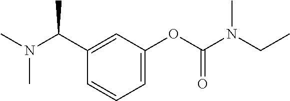 | 35.5 |
| 1A | Physostigmine | 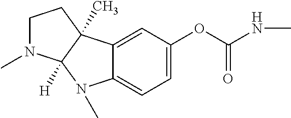 | 0.07 |
| 2 | S-riva tranylcypromine | 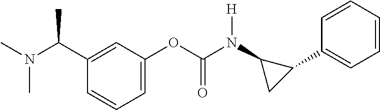 | 0.2 |
| 3 | S-riva- amoxapine | 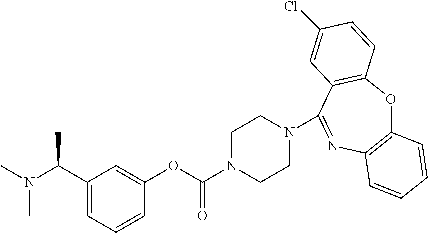 | 20.9 |
| 4A | S-riva- desipramine | 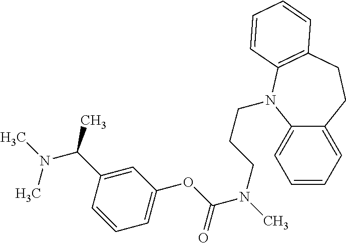 | 0.2 |
| 5A | S-riva- nortriptyline | 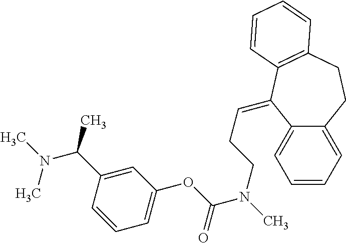 | 0.5 |
| 6A | S-riva- protriptyline | 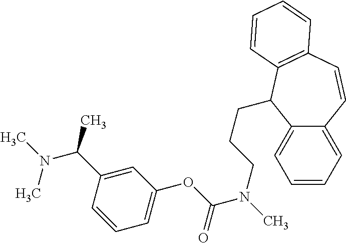 | 0.5 |
| 7A | S-riva fluoxetine | 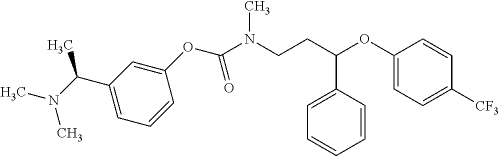 | 4.8 |
| 8A | S-riva fluvoxamine | 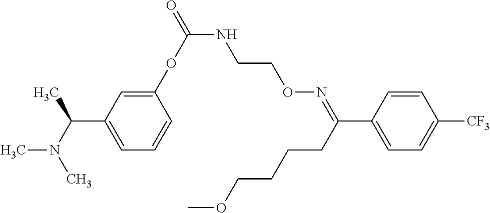 | 6.1 |
| 9A | S-riva paroxetine | 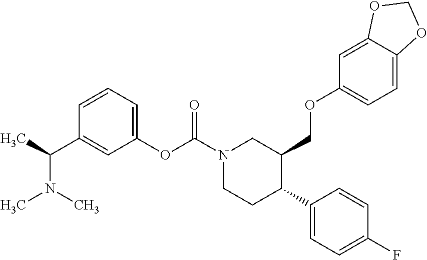 | 9.5 |
| 10A | S-riva duloxetine | 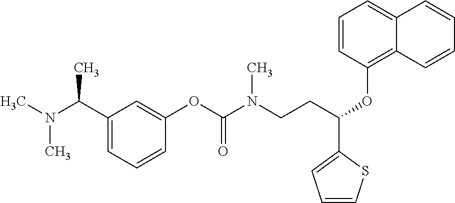 | 0.2-0.5 |
| 16 | S-riva propylhexedrine | 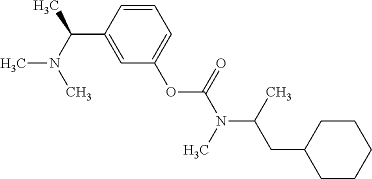 | 0.9 |

These data show that the carbamoyl esters of the invention inhibit acetylcholinesterase in vitro. Inhibition of acetylcholinesterase by carbamoyl esters can be greater than inhibition of acetycholinesterase by a stigmine, such as rivastigmine. Carbamoyl esters synthesized from stigmines resulted in similar or increased activity compared to the stigmine. For example, the carbamoyl ester (14) resulted in a 10 fold increase in enzymatic activity compared to rivastigmine. Thus, structural alterations in stigmines, carbamoyl esters with known enzymatic activity, did not decrease or inhibit the enzymatic activity of the stigmine.

### Example 10

### Compounds of the Invention Inhibit Cholinesterase in Brain

Male Wistar rats were injected intraperitoneally (i.p.) with rivastigmine or with compounds of the invention. The dose of rivastigmine or carbamoyl ester resulted in a cholinergic behavioral effect with minimal side effects and was well-tolerated by the animals. Animals were decapitated 3 hours after injection and the brains rapidly removed. The brain tissue was diced into small pieces, placed on ice and immediately homogenized with a Polytron PT1200 (Kinematic AG) in 10 ml ice cold Tris with 0.1% Triton-X and protease inhibitors. The protease inhibitors in the extraction buffer were Antipain (10 _M), Aprotinin (5 TIU/mg protein), Bestatin (60 nm), Leupeptin (10 _M) and Pepstatin (1 _M). The final dilution of the homogenate in the final assay mixture was 120-fold.

Total cholinesterase activity was determined by a modification of the colorimetric method of Ellmann, et al. (Biochem. Pharmacol., 7:88-95 (1961)), as described above. Hydrolysis of acetylthiocholine was monitored indirectly by measurement of the formation of the conjugate between thiocholine and DTNB. Optical density at 405 nm was recorded during five (5) minutes employing a a microplate spectrophotometer (Polarstar, BMG Labtech), and plotted against time. The initial rates were calculated from the slope of the linear portion of the graph.

Cholinesterase activity was normalized for protein content of the homogenate. Relative cholinesterase activity was calculated as the ratio of normalized cholinesterase activity in a rat treated with a control compound or a carbamoyl ester over normalized cholinesterase activity in saline treated rats.

These data are summarized below:

| | | Relative ChE | |
|---|---|---|---|
| Compound | dose | Activity | ChE inhibition |
| Rivastigmine | 2 mg/kg | 85% | 15% |
| 7 | 2 mg/kg | 62% | 38% |
| 9 | 8 mg/kg | 59% | 41% |

These data show that systemic administration of compounds of the invention results in inhibition of total cholinesterase activity in the brain of mammals. The carbamoyl esters resulted in significantly increased inhibition of cholinesterase activity in the brain compared to rivastigmine with minimal side effects. Thus, the carbamoyl esters of the invention can be employed in methods that inhibit cholinesterases with few side effects compared to currently available cholinesterase inhibitors.

### Example 11

### In Vitro Screening of Compounds of the Invention

An in vitro screening assay with various conjugates was completed according to the methods described in Ellman G L et al., Biochem Pharmacol., 7:88-95 (1961) and in Nadarajah B, J. Anal. Toxicol., 16:192-193 (1992), both of which are herein incorporated by reference in their entireties. The assay method was completed according to the following:

| | |
|---|---|
| Source | Human recombinant HEK-293 cells |
| Substrate | 700 μM acetylthiocholine |
| Vehicle | 1% DMSO |
| Pre-Incubation | 15 minutes at 25° C. |
| Time/Temp. | |
| Incubation Time/Temp. | 20 minutes at 25° C. |
| Incubation Buffer | 0.1 M sodium phosphate, pH 7.4 |
| Quantitation Method | Spectrophotometric quantitation |
| | of thiocholine |
| Significance Criteria | ≧50% of max stimulation or inhibition |

The assay results are summarized below:

| | | | BuChEI | BuChE | BuChEI |
|---|---|---|---|---|---|
| | Cmpd | AChEI (%) | (%) | I (%) | (%) |
| Carbamoyl ester | No. | 10 μM | 1 μM | 10 μM | 1 μM |
| S-rivastigmine | | 19 | | 100 | 45 |
| Phenserine | | 99 | 93 | 42 | |
| S-rivastigmine- | 9 | 79 | 35 | 90 | 27 |
| 1-amphetamine | | | | | |
| S-rivastigmine-d- | 20 | 76 | 35 | 20 | |
| amphetamine | | | | | |
| S-rivastigmine-1- | 7 | 79 | 32 | 17 | |
| methamphetamine | | | | | |
| S-rivastigmine-d- | 13 | 97 | 51 | 95 | 25 |
| methamphetamine | | | | | |
| Physostigmine-d- | 14 | 94 | 45 | 28 | |
| amphetamine | | | | | |
| S-rivastigmine- | 15 | 95 | 49 | 30 | |
| methoxyphenamine | | | | | |
| S-rivastigmine- | 11 | 52 | 13 | 17 | |
| desmethylselegiline | | | | | |
| R-rivastigmine- | 17 | 31 | | 12 | |
| desmethylselegiline | | | | | |
| Physostigmine- | 18 | 37 | | 8 | |
| desmethylselegiline | | | | | |
| S-rivastigmine- | 2 | 97 | 84 | 67 | 23 |
| tranylcypromine | | | | | |
| S-rivastigmine- | 5 | 89 | 29 | 14 | |
| atomoxetine | | | | | |
| S-rivastigmine- | 3 | 16 | | 6 | |
| amoxapine | | | | | |
| S-rivastigmine- | 4A | 99 | 91 | 60 | 16 |
| desipramine | | | | | |
| S-rivastigmine- | 5A | 99 | 81 | 55 | 24 |
| nortriptyline | | | | | |
| S-rivastigmine- | 6A | 99 | 81 | 53 | 16 |
| protriptyline | | | | | |
| S-rivastigmine- | 7A | 76 | 11 | 15 | |
| fluoxetine | | | | | |
| S-rivastigmine- | 8A | 76 | 17 | 6 | |
| fluvoxamine | | | | | |
| S-rivastigmine- | 9A | 69 | 9 | 4 | |
| paroxetine | | | | | |
| S-rivastigmine- | 10A | 100 | 88 | 36 | |
| duloxetine | | | | | |

### Example 12

### Measurement of Hypothermia and Determination of the Dose Range and Time Course for Cholinergic Effects

The induction of hypothermia was determined for compounds of the invention according to the methods described in Freedman, et al., European Journal of Pharmacology, 187 (1990), 193-199, which is incorporated by reference herein. Hypothermia is a marker of CNS penetration for AChE inhibitors.

The dose range and time course for cholinergic effects of the compounds of the invention was determined as described below.
Subjects: Two hundred eight male CD IGS (Sprague Dawley derived) rats were received at 126-150 grams and maintained four per cage on a regular light/dark cycle (lights on 0600-1800) with ad libitum food and water for about 1 week before commencement of experimentation.
Apparatus: Injections were made subcutaneously (s.c.) with a 25-gauge needle on a 1-mL tuberculin syringe. Observations were made in a 5½×10-inch polycarbonate rat housing cage. Temperature was taken with a rat rectal probe on a Model BAT-12 electronic thermometer.
Compound Preparation: Test compounds were dissolved for example, in 0.9% saline. Concentrations for lower doses were prepared by taking aliquots from higher concentrations and diluting. Injection volumes were 1 mL/kg, if the test compound was sufficiently soluble. If less soluble, maximum injection volume were 5 mL/kg. Route of administration was s.c. A sample protocol is as follows:
Treatment Groups included (N=3, with 6 for Saline)
   Saline
   (S)-Rivastigmine at 1, 3, 10, 30, and 100 mg/kg
   (R)-Rivastigmine at 1, 3, 10, 30, and 100 mg/kg
   Test Compounds

Test Compounds were dosed at 1, 3, 10, 30, and 100 mg/kg
Procedure: The rats were brought to the test room in the home cage. Baseline temperatures were measured just before injection. After injection s.c., the rat was placed in the observation cage. At 0.5, 1, 2, and 4 hours after injection, it was observed briefly for gross signs; salivation was scored as absent, clearly present, or copious; and rectal temperature was taken. In observation for gross signs, special attention was paid to fasciculation (muscle twitch), tremor/ataxia, and abnormal gait. The experiment was designed to allow assessment of one rat per time point per minute. Salivation score and temperature were determined within this constraint, but only the most salient of gross signs were noted. After the 4-hour observation point, or sooner if signs of distress were observed, the rat was euthanized by CO₂ inhalation.
Data Analysis: Gross signs, salivation score, and temperature at each time point were tabulated for inspection. This is a combination of within-subject design for control and between-subjects design for dose effect. That is, the effect of the compound was measured against the reading taken immediately before injection, and the difference between doses of the compound was measured between groups of three rats. As a precaution against the possibility of large effects caused by repeatedly measuring rectal temperature, a vehicle group (N=6) was included in the pilot with (s)-rivastigmine.
   Results of the hypothermia and dose determination are shown below.

| | | | Hypothermia | Max | |
|---|---|---|---|---|---|
| Cmp | | | MED | Tolerated | TIMTD/ |
| # | Name | Compound Structure | (mg/kg) | Dose | MED |
| 1 | S- rivastigmine | 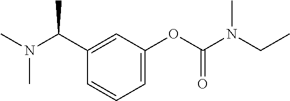 | ≦0.1 | 10 | ≧100 |
| 3 | S-riva- amoxapine | 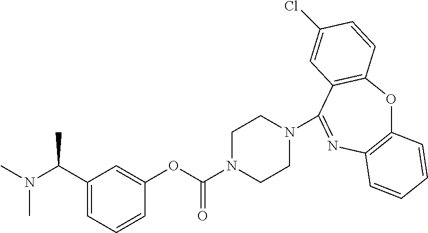 | 10 | ≧100 | ≧10 |
| 4A | S-riva- desipramine | 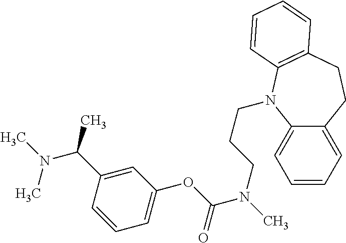 | 3 | ≧100 | ≧30 |
| 5A | S-riva- nortriptyline | 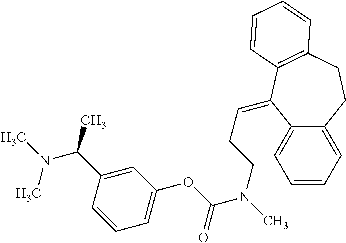 | ≦1 | ≧100 | ≧100 |
| 6A | S-riva- protriptyline | 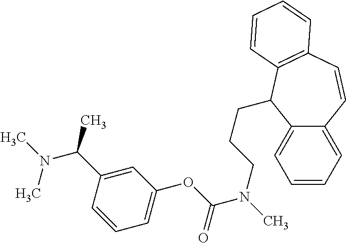 | 100 | >100 | >1 |
| 2 | S-riva- tranylcypromine | 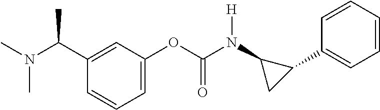 | 10 | 30 | 3 |
| 7A | S-riva- fluoxetine | 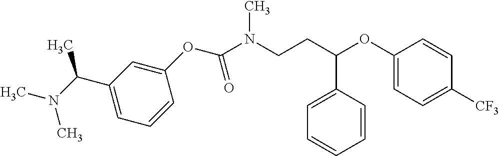 | 3 | ≧100 | ≧30 |
| 16 | S-riva- propyl- hexedrine | 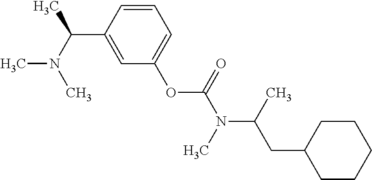 | 10 | ≧100 | ≧10 |

### Example 13

### Determination of Compounds for Abuse Potential and for Use to Treat or Prevent Substance Abuse

Compounds of the invention are tested for abuse potential and for the potential to treat drug abuse using methodology that is well known in the art. See, for example review by Bergman and Paronis, Molecular Interventions, October 2006, Vol. 6, Issue 5, p. 273-283. Compounds are tested to determine if the compounds are “self-administered” in monkeys. Self-administration is a contingency arrangment under which responding is controlled by the delivery of a unit dose of drug. Common drugs of abuse, such as cocaine, amphetamine, mu opioid agonists, and sedative hypnotics are “self-administered.”

The invention includes a method for the treatment or prevention of a substance use disorder, wherein there is a decrease in self-administration of the abused substance demonstrated in an animal model. In one aspect, self-administration of the abused substance is decreased and the ability of the subject to obtain a reinforcer is not altered. A reinforcer is a stimulus event that increases the ability of an individual to respond. In one aspect, the reinforcer is a drug of abuse. In one aspect, the decrease in self-administration is across a broad range of abused substance doses. The decrease in self-administration is dose-dependent. In one aspect, the decrease in self-administration is sustained over time.

Following testing of the compounds for inherent abuse potential, the compounds are tested to determine whether chronic or subchronic treatment reduces the self-administration in monkeys of standard abused drugs such as cocaine, amphetamine, and mu opioid agonists. Compounds of the invention that reduce self-administration in monkeys are useful to treat the condition of drug addiction.

### Example 14

### Drug Discrimination Using a Self-Administration Test

A drug discrimination study was conducted using a well known in the art self-administration test, See, for example: Bergman and Paronis, Molecular Interventions, October 2006, Vol. 6, Issue 5, p. 273-283, (Graboski, et al., Agonist-like, replacement pharmacotherapy for stimulant abuse and dependence, Addict Behav 29(7):1439-1464, 2004; Negus and Mello, Effects of chronic d-amphetamine treatment on cocaine- and food-reinforced responding under a second-order schedule in rhesus monkeys, Drug Alcohol Depend 70(1):39-52, 2003; Negus and Mello, Effects of chronic d-amphetamine treatment on cocaine-and food-maintained responding under a progressive-ratio schedule in rhesus monkeys, Psychopharmacology (Berl) 167(3): 324-332, 2003).

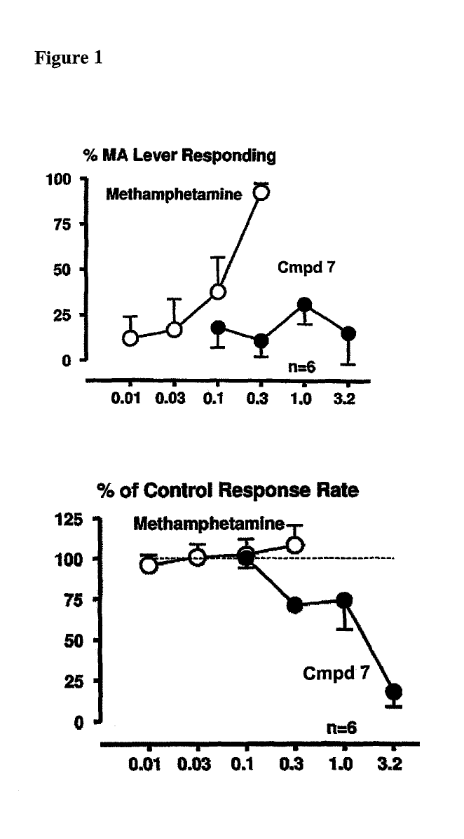

In the drug discrimination study, rats were trained as follows: after injection of methamphetamine, pressing one lever produced food reward; after injection of vehicle, pressing the other lever produced food reward. In animals trained in this way to discriminate methamphetamine from vehicle, on test days they received an injection of compound 7, and pressing either lever now produced food reward. Testing was performed to determine whether rats selected the “methamphetamine lever” or “vehicle lever” after being administered compound 7. Compound 7 was dosed to rats as follows: 0.01, 0.03, 0.1, 0.3, 1.0, and 3.2 mg/kg i.p. and responding on the methamphetamine and vehicle levers was recorded as shown in FIG. 1. The result of the drug discrimination test shows a lack of stimulus generalization to methamphetamine in rats treated with compound 7. Compound 7 showed no generalization to methamphetamine up to doses that markedly decreased reponse rates.

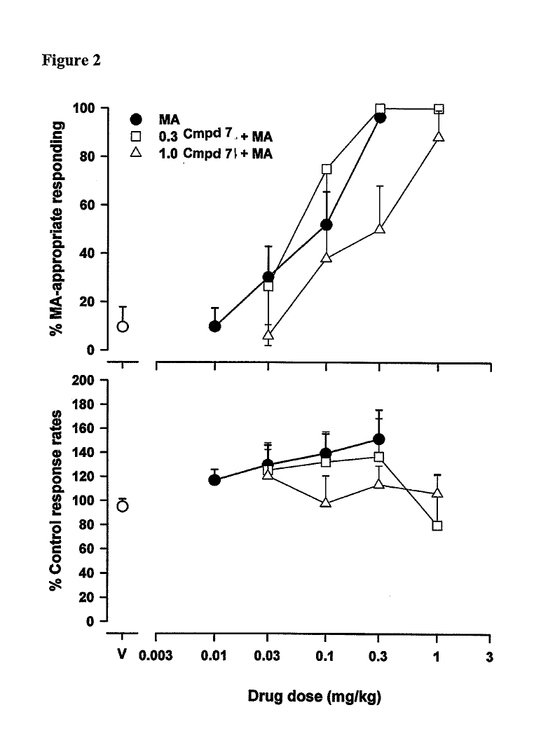

FIG. 2 shows data also from a methamphetamine drug discrimination test in rats. The data in the top figure shows that a dose of 1.0 mg/kg of compound 7 shifted the methamphetamine dose-response curve to the right. These data indicate that compound 7 could block the discriminative effects of methamphetamine, and this was accomplished without behavioral disruption as shown in the lower figure. These data suggest that compound 7 may be an effective treatment for methamphetamine-like stimulant abuse.

### EQUIVALENTS

While this invention has been particularly shown and described with references to preferred embodiments thereof, it will be understood by those skilled in the art that various changes in form and details may be made therein without departing from the scope of the invention.

1. A method for treating or preventing a substance use disorder in an individual comprising administering to the individual a compound having the formula: 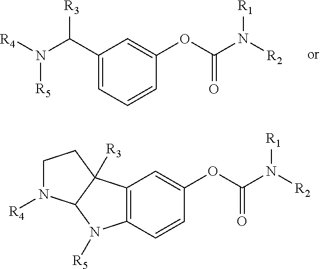
or a pharmaceutically acceptable salt thereof, wherein
R₁ is selected from the group consisting of hydrogen, unsubstituted alkyl, and substituted alkyl;
R₂ is selected from the group consisting of substituted alkyl, unsubstituted aralkyl, substituted aralkyl, unsubstituted (heterocycle)alkyl, substituted (heterocycle)alkyl, unsubstituted heteroaralkyl, substituted heteroaralkyl, unsubstituted aryl, substituted aryl, unsubstituted heteroaryl, substituted heteroaryl, unsubstituted cycloalkyl, substituted cycloalkyl, unsubstituted heterocycloalkyl and substituted heterocycloalkyl;
or taken together with the nitrogen atom to which they are attached, R₁ and R₂ form a 5- or 6-membered ring, further wherein the ring is substituted or unsubstituted;
R₃ is selected from the group consisting of hydrogen, unsubstituted alkyl, and substituted alkyl;
R₄ is selected from the group consisting of hydrogen, unsubstituted alkyl, and substituted alkyl; and
R₅ is selected from the group consisting of hydrogen, unsubstituted alkyl, and substituted alkyl.

2. A method of treating or preventing a substance use disorder in an individual comprising administering to an individual a compound having the formula: 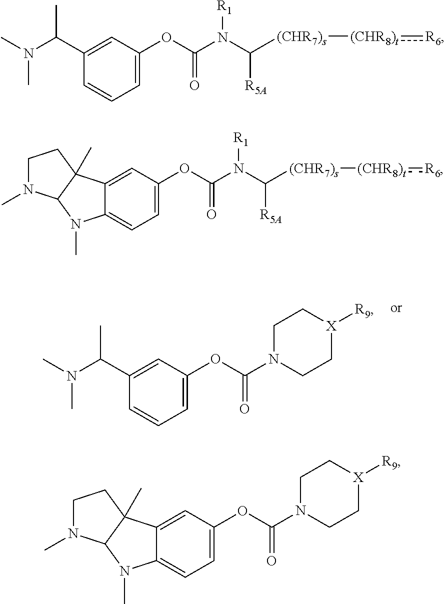
or a pharmaceutically acceptable salt thereof, wherein
R₁ is selected from the group consisting of hydrogen, unsubstituted alkyl, and substituted alkyl;
R_{5A} is selected from the group consisting of hydrogen, unsubstituted alkyl, and substituted alkyl; and
R₆ is selected from the group consisting of unsubstituted aryl, substituted aryl, unsubstituted cycloalkyl, substituted cycloalkyl, unsubstituted tricyclic ring, and substituted tricyclic ring;
R₇ is selected from the group consisting of hydrogen, unsubstituted alkyl, and substituted alkyl;
R₈ is selected from the group consisting of hydrogen, unsubstituted alkyl, substituted alkyl, substituted aryloxy, unsubstituted aryloxy; and
s is 0 or 1;
t is 0 or 1, provided that s and t are not both 0; and
- - - is absent or taken together with the bond shown directly above it forms a double bond;
X is N or CH;
R₉ is selected from the group consisting of hydrogen, substituted tricyclic ring, unsubstituted tricyclic ring, substituted aryl, unsubstituted aryl; and further wherein the piperidine and piperazine ring is optionally substituted.

3. The method according to claim 1 comprising the compound or pharmaceutically acceptable salt thereof, wherein R₃, R₄, and R₅ are unsubstituted alkyl.

4. The method according to claim 1 comprising the compound or pharmaceutically acceptable salt thereof, wherein R₁ is hydrogen or unsubstitued alkyl.

5. The method according to claim 1 comprising the compound or pharmaceutically acceptable salt thereof, wherein R₂ is unsubstituted or substituted aralkyl.

6. The method according to claim 5 comprising the compound or pharmaceuticall acceptable salt thereof, wherein R₂ is

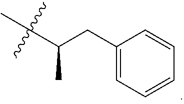

7. The method according to claim 1 or 2, wherein one or more side effects are decreased or eliminated, wherein said side effect is selected from behavioral toxicity, insomnia, sleep disturbance, muscle twitching, cardiovascular responses (increased blood pressure and increased heart rate), thermoregulation problems, paranoia, hallucinations, dependence or pseudo-addiction are decreased or eliminated.

8. (canceled)

9. (canceled)

10. The method according to claim 1 or 2, wherein the substance associated with the disorder is selected from cocaine, amphetamine or amphetamine-like substance e.g., dextroamphetamine, nicotine, mu opioid agonists e.g., morphine, buprenorphine, fentanyl, levorphanol, meperidine, and methadone, and dextrorphan, and combinations of the above.

11. The method according to claim 1 or 2, wherein the substance use disorder is selected from drug withdrawal disorder, amphetamine withdrawal disorder, cocaine withdrawal, nicotine withdrawal, opioid withdrawal, and withdrawal symptoms due to addictive substances.

12. (canceled)

13. (canceled)

14. The method according to claim 1 or 2, wherein the compound or pharmaceutically acceptable salt thereof is administered enterally, parenterally, orally or intramuscularly.

15. The method according to claim 1 or 2, wherein the compound or pharmaceutically acceptable salt thereof is administered chronically.

16. (canceled)

17. (canceled)

18. (canceled)

19. A kit for carrying out the method according to claim 1 or 2.
